(19) United States
(12) Patent Application Publication
(10) Pub. No.: US 2024/0261085 A1
(43) Pub. Date: Aug. 8, 2024

(54) TOOLS AND METHODS FOR GRAFT DEPLOYMENT

(71) Applicant: Sparta Biopharma Inc., Madison, NJ (US)

(72) Inventors: Adam W. MARTINEZ, Madison, NJ (US); Travis E. ENTROP, Madison, NJ (US)

(21) Appl. No.: 18/554,194

(22) PCT Filed: Apr. 5, 2022
(86) PCT No.: PCT/US2022/023469

§ 371 Date: Oct. 5, 2023

Related U.S. Application Data
(60) Provisional application No. 63/170,904, filed on Apr. 5, 2021.
(60) Provisional application No. 63/315,080, filed on Feb. 28, 2022.

(51) Int. Cl. A61F 2/08; A61B 17/04

(52) U.S. Cl. CPC A61F 2/0805; A61B 17/0401; A61F 2/0811; A61B 2017/0409; A61B 2017/0445; A61F 2002/0841; A61F 2002/0876

(57) ABSTRACT

Described herein are methods and apparatuses (e.g., systems and devices, including tools) for delivering and securing grafts in a patient's body, especially connective tissue-to-bone interface grafts for repair of the enthesis, including ligament to bone and tendon to bone interface scaffolds for repair of torn tendons, ligaments, and/or calcified and uncalcified fibrocartilage.

### CLAIM OF PRIORITY

This patent application claims priority to U.S. provisional patent application No. 63/170,904, titled “TOOLS AND METHODS FOR GRAFT DEPLOYMENT,” filed on Apr. 5, 2021 and U.S. provisional patent application No. 63/315,080, titled “TOOLS AND METHODS FOR GRAFT DEPLOYMENT,” and filed on Feb. 28, 2022, each of which is herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### BACKGROUND

Connective tissues, including tendons and ligaments, provide joint stability, guide joint motion, and play an important role in proprioception. Injury to ligaments and tendons represents a major portion of all sports-related injuries on an annual basis. In particular, rotator cuff injuries are particularly prevalent and difficult to repair.

The rotator cuff is a group of muscles and tendons that surround the shoulder joint, keeping the head of your upper arm bone firmly within the shallow socket of the shoulder. A rotator cuff injury can cause a dull ache in the shoulder, which often worsens when you try to sleep on the involved side. Rotator cuff injuries occur most often in people who repeatedly perform overhead motions in their jobs or sports. Examples include painters, carpenters, and people who play baseball or tennis, although rotator cuff tears may occur as a result of a single injury. The risk of rotator cuff injury increases with age.

Extensive rotator cuff tears typically require surgical repair, however current techniques are not optimal, and may be difficult or impossible to achieve, or may fail days or months after surgery. In particular, these regions are difficult to access, and even when accessed may be difficult to operate within. Although grafts and repair techniques are known, it has proven particularly difficult to implant or insert these typically flat (e.g., postage-stamp or larger) sized implants, which may be somewhat delicate, as they may be include biological (e.g., bone) material. Current standard of care for reattaching connective tissues (ligament, tendon) to bone typically uses suture and suture anchors to reattach the connective tissue to the bone. Re-tearing of the connective tissue is common and occurs in over half of the cases. Further, full mobility and pain relief are not generally possible.

Thus, there is a need for tools and methods for implanting connective tissue-to-bone interface grafts. Further, there is a need for methods and tools for delivering and securing such graft to the bone.

### SUMMARY OF THE DISCLOSURE

Described herein are tools and methods for delivering and securing graft implants in a patient's body, especially connective tissue-to-bone interface grafts for repair of the enthesis, including ligament to bone and tendon to bone interface scaffolds for repair of torn tendons, ligaments, and/or calcified and uncalcified fibrocartilage.

Described herein are methods and apparatuses for repairing tissue, including, but not limited to repairing an enthesis. Also described herein are methods and apparatuses for delivering and securing graft implants to tissue at a target location within a patient's body. The apparatuses (e.g., devices and/or systems, including tools) and methods described herein are well suited for use in the delivery and implantation of grafts between connective tissue, such as ligament, and bone. For example, in some implementations, the apparatuses and methods described herein are used in the delivery and implantation of connective tissue-to-bone interface scaffolds (e.g., ligament-to-bone interface scaffolds, tendon-to-bone interface scaffolds, etc.).

The apparatuses and methods described herein may involve temporary fixation of a graft at the site of repair, for example, to prevent movement of the graft while the graft is being permanently secured to the site of repair between the bone and connective tissue. Temporary fixation may involve placement of a retention component, such as a removable securement (e.g., a suture, removable anchor, removable pin, removable staple, etc.) that is configured to temporarily hold the graft in place only until the connective tissue (e.g., ligament) is secured over the graft, so that the graft is sandwiched securely between the connective tissue and a region of decorticated bone. Once the graft is secured between the connective tissue and decorticated bone, the removable securement can be removed from the graft, and from the patient's body, thereby minimizing concerns of foreign body response or tissue damage (e.g., compared to a long-term implanted component). Such techniques can also reduce surgical complexity and avoid time-intensive biocompatibility characterization and mechanical validation of using long-term implanted components. In particular, these methods and apparatuses allow a graft to be secured (including permanently secured) in place between connective tissue (e.g., ligament) and decorticated bone without any other material (e.g., suture, pin, staple, etc.) disposed between the connective tissue and graft or bone and graft, which is a significant benefit in reducing the likelihood of complications and post-surgical discomfort and may reduce healing times.

The apparatuses described herein include removable securements, which in some configurations comprises removable sutures and/or pins and/or staples. The apparatuses described herein also include removable securement delivery tools for delivering one or more removable securements. A removable securement delivery tool may be configured as a puncture tool configured to form small holes within the bone, which may be referred to herein as marrow vents or microfractures, deep enough to access and allow bone marrow material to migrate into the graft positioned over the vents or microfractures. Transfer of marrow into the graft may increase endogenous growth factors and stems cells at the site of graft implantation, which may have a therapeutic benefit and promote healing. The removable securement delivery tools described herein may also be adapted to deliver one or more removable securements to temporarily hold the graft in place.

Although the methods and apparatuses (e.g., devices and systems, including tools) can be used during any of a number of graft implant procedures, they may be especially well suited for use during implantation of connective tissue-to-bone interface grafts (e.g., scaffolds). Such connective tissue-to-bone interface grafts may be used to repair of ligaments, tendons, cartilage or growth plates in the shoulder, hand, elbow, knee, foot, ankle or any other anatomical location as needed. Furthermore, the grafts may be applied to replace or repair any of a variety of joints. Any appropriate tissue may be repaired, including, but not limited to rotator cuff tissue.

The connective tissue-to-bone interface grafts may have a porous structure to provide a large surface area for exchange of nutrient and waste. In some examples, the grafts may be formed of a bone material that has been processed to form a layered structure having a first porous layer that is demineralized and a second porous layer that is mineralized. The graft may be a strip, having an elongate, flattened sheet or strip-like configuration, in which the first (e.g., upper) major side is mineralized and the second (e.g., lower) major side is demineralized. The edges between the first and second side (minor sides) may be rounded or beveled. The demineralize layer thickness may be larger than the thickness of the mineralized layer (e.g., 60% or greater, 65% or greater, 70% or greater, 75% or greater, etc. of the thickness of the graft).

The cortical layer may be removed from the implantation site for the graft prior to removably attaching the graft. In addition, microfractures may be formed in the bone, to further expose the marrow. For example, for a humeral procedure, the humerus may decorticated and alternatively or additionally microfractures formed at the site onto which the graft is to be applied. For example, the bone (e.g., humerus) may be prepared by removing a region of the cortical layer, e.g., to form a shelf or landing pad region to which the graft may be attached. Alternatively or additionally, in some examples the bone (e.g., humerus) may be prepared by forming microfractures to the region to which the graft will be attached, without removing a region. In some examples both a region of the bone may be removed, and the bone may be treated to form microfractures.

For example, a method of repairing a tissue (e.g., a method of repairing a rotator cuff) may include: removing a region of a cortical layer of a bone (e.g., a humerus) to form a decorticated surface; positioning a graft over the decorticated surface; temporarily securing a mineralized face of the graft on the decorticated surface with a removable securement, wherein the mineralized face has a thickness that is less than a demineralized face of the graft that is adjacent to the mineralized face; suturing a tendon against the demineralized face of the graft with one or more sutures that do not pass through the graft; and removing the removeable securement from the graft so that the graft is held against the decorticated surface by just the surgically repaired tendon. The surgical repair of the tendon to bone provides the retention force after the removeable securement is removed.

As mentioned, in general, the removable securement may be a retention device. Thus, the removable securement may include one or more penetrating arms, e.g., prongs, and a tether (e.g., “pull suture”) that may allow the removable securement to be removed (or partially removed) so that it is not between the graft and the tendon or the graft and the bone. For example, the removable securement may include one or more prongs for penetrating into the bone, and in particular, the decorticated bone. Thus, any of these methods may include passing one or more prongs of the removable securement through the graft and into the decorticated surface.

In general, the removable securement may be removed without disturbing the tendon or tendon fixation system (i.e. suture), which is attached over the graft, to the bone. For example, removing the removable securement may include removing the removable securement from between the graft and the tendon, leaving the tendon sutured against the graft.

In some examples, temporarily attaching the graft may also beneficially add one or more openings for the passage of marrow cells from the bone into the graft; this may be done in addition to decorticating the bone. For example, any of these methods may include temporarily securing the graft by driving one or more prongs of a removeable securement through the decorticated surface to create one or more marrow vents within the humerus into a marrow material. Removing the removeable securement may include removing the one or more prongs from the decorticated surface, thereby causing the marrow material to exit the one or more marrow vents and into the graft. In some examples removing the removeable securement comprises removing the one or more prongs from the decorticated surface by pulling a tether (e.g., pull suture) attached to the removeable securement. Pulling the tether may include pulling the pull suture in a non-perpendicular direction with respect to a long axis of the one or more prongs. In some examples, pulling the tether causes the removeable securement to pivot such that the one or more prongs exit corresponding one or more opening within the decorticated surface.

Any of these methods may include forming microfractures in the decorticated surface cither before or during removably attaching the removable securement. A separate tool may be used to form microfractures, or the same tool used to removably attach the removable securement may be used.

Any of these steps may be performed minimally invasively, including arthroscopically, e.g., through a cannula or introducer. For example, decorticating the bone surface may be done arthroscopically (e.g. using a burr or other tool). The graft, and in particular, the flexible grafts described herein (including those described in U.S. patent application Ser. No. 17/015,043, titled “CONNECTIVE TISSUE TO BONE INTERFACE SCAFFOLDS,” filed on Sep. 8, 2020, herein incorporated by reference in its entirety) may be inserted arthroscopically, and positioned over the decorticated surface. The step of temporarily securing the graft on the decorticated surface with a removable securement may be done minimally invasively as well, including using any of the tools described herein to temporarily secure the graft. The tendon may be sutured to the bone using arthroscopic techniques, including placing the anchors (e.g., suture anchors) to the bone. Finally, the removeable securement may be removed from the graft so that the graft is held against the decorticated surface by just the surgically repaired tendon by a minimally invasive (e.g., arthroscopic) technique.

The methods described herein may also include (in some examples when suturing the tendon) anchoring the one or more tendon-securing sutures to the humerus outside of the decorticated region. It may be beneficial to anchor the tendon-securing sutures via one or more anchors that are outside of the decorticated region, and also separate from the graft. The tendon may be secured in place by any appropriate means, e.g., using two or more sutures that may, e.g. cross over the tendon one or more times.

Any of the methods described herein may be methods of repairing an enthesis.

In general, these methods may be used to treat any appropriate tissue, including (but not limited to) shoulder and knee tissues. For example, suturing the tendon may include suturing the tendons of one or more of: the supraspinatus muscle, infraspinatus muscle teres minor muscle or the subscapularis muscle.

The methods described herein may be performed as part of a single, acute procedure. Thus, the removable securement may be removably attached through the graft, the connective tissue (e.g., tendon) secured to the bone and the removable securement removed, all during the same procedure. For example, the removable attachment may be removed from the bone within less than 2 hours of attaching it through the graft, e.g., less than 1.5 hours, less than 1 hour, less than 45 minutes, less than 30 min, etc.

Also described herein are removable securement delivery tools. These tools may be used to secure the graft, temporarily, to the decorticated bone using a removable securement, and/or to assist in removing the removable securement. In some examples, the removable securement may be loaded into the removable securement delivery tool, or multiple removable securements may be loaded to the tool. The tool may direct the tether coupled to the one or more securements so that they do not get entangled.

For example, a removable securement delivery tool may include: a handle; and an elongate body attached to a proximal handle, a distal end of the elongate body including a delivery portion configured to deliver a removeable securement into a bone, wherein the deliver portion includes one or more pins adapted to fit through and engage with one or more tubular posts (e.g., prongs) of the removeable securement, each of the one or more pins including a sharp distal tip configured to penetrate bone and create a microfracture hole within the bone, wherein the delivery portion is adapted to release the removeable securement such that the one or more tubular posts of the removeable securement remain within the one or more microfracture holes (until the removable securement is removed).

The delivery portion may include an engagement surface that is adapted to engage with a flange portion of the removeable securement. The one or more pins may be configured to translate relative to the engagement surface, e.g., so that they can be removed from the removable engagement, e.g., removed from out of the hollow, and in some cases cylindrical, posts or prongs of the removable securement. The one or more pins may include a sharp distal end that may penetrate the bone (e.g., the decorticated bone region).

The side of the elongate body of the tool may include one or more channels configured to accommodate the tether of the removeable securement. The channel may be recessed (as a groove) and may be open or partially open, or it may be enclosed.

In some examples the delivery portion of the removable securement delivery tool is specifically configured or adapted for use with a particular type of removable securement. For example the delivery portion may include one or more pins adapted to fit through and engage with one or more tubular posts (prongs) of the removeable securement. For example, in some cases the delivery portion includes two pins adapted to fit through and engage with two tubular posts (prongs) of a removeable securement.

Also described herein are removeable securements that are adapted for use during a repair procedure (e.g., rotator cuff repair procedure). The removeable securement may include: a flange having a substantially flat surface; one or more tethers coupled to the flange; and one or more posts extending proud from the flange, each of the one or more posts having a central opening extending therethrough, wherein the outer surface of the one or more posts is adapted to releasably engage with a decorticated bone. The tether may be a suture or other material, including polymeric, metallic, or natural (e.g., fibers) that have sufficient strength to allow the removable securement to be removed by pulling on the tether without the tether breaking. For example the tether may be made of a synthetic non-absorbable material. In some examples the tether has a diameter of between about 0.01 mm and 1 mm (e.g., between about 0.1 mm and about 0.8 mm, etc.).

The flange region may be configured to prevent the removable securement from penetrating too deeply into the graft and bone. The flange region also prevents the graft from pulling over the removable securement. In some cases the flange region has a surface area that is sufficiently large to prevent damage to the graft during insertion. For example, the flange region, which may also be referred to as a head region, may have an area that is approximately 4× the cross-sectional area of the one or more prongs in a plane parallel with the primary plane of the flange region. (e.g., parallel with the graft, when inserted). The flange may be rounded.

The flange region may be flat (e.g., substantially planar, particularly on the graft-facing surface, or it may be curved). The flange region may extend in a plane that is substantially perpendicular to the direction of the prongs, or in some cases, at an angle of between about 100 and 160 degrees, relative to the prongs; in some case this angle may making removing the removable securement easier, particularly when removing the removable securement from underneath of the tendon at an angle.

The prongs (posts) may be configured so that they may be removed by applying a reasonably low force to the tether to remove them from the tissue. In some examples the one or more prongs (posts) may have a smooth outer profile. The outer profile may be tapered. In some examples the prongs (posts) may be relatively smooth, and/or may be formed of and/or coated with a lubricious material (e.g., Teflon, silicone, etc.). In some cases the prongs may be treated with a dissolvable and/or collapsible material that may reduce the force required to remove the prongs (posts) over the short period of time they are in the body during the procedure (e.g., within 1 minute, 2 minutes, 3 minutes, 4 minutes, 5 minutes, etc.).

In some examples the flange has a C-shape or a U-shape; in some examples the flange has an oval shape or a half-moon shape. The removable securement may include one or more openings to which the one or more tethers are coupled. In some examples, the tether is coupled to the flange region. In some examples the flange is coupled to an extension or projection of the flange region.

The flange region may be relatively thin. For example, the flange region may have a transverse thickness of less than about 2 mm, less than about 1.5 mm, less than about 1 mm, etc.

In some examples, the prongs (posts) are configured to penetrate the graft, but not penetrate more than shallowly into the bone (e.g., the decorticated bone). For example, for grafts that are between about 2 mm and about 8 mm thick, the prongs (posts) of the removeable securement may each have a length of between about 3 and about 9 mm (e.g., from about 3.5 mm to 10 mm, etc.). In some examples the removable securement dimensions may be matched to the dimensions (thickness) of the graft to be inserted. In some examples the length of the prong (post) may be between about 0.5 and about 4 mm (e.g., between about 0.5 and about 3 mm, between about 0.5 and 2 mm, etc.) longer than the thickness of the graft. In some cases the prongs (posts) may have a diameter or between about 0.8 mm and 3 mm (e.g., between about 0.8 mm and 2 mm, between about 0.8 mm and 1.5 mm, etc.) on average. In some cases the prongs/posts may be tapered along at least the bone-penetrating length of the prong, which may make them easier to remove when pulling the tether. For example, the distal end region of the prong (post) may be tapered over the last few mm.

Although many of the examples described herein are configured to remove the entire removable securement, in some examples the removable securement may be configured to break in a controlled manner so that a small portion (e.g., the portion embedded in the bone) is left behind, while the rest of the removable securement is removed. For example, the removable securement may be configured so that the one or more prongs (posts) are configured to include a frangible (breakable) region along the length, particularly within the distal end region, when force is applied by pulling the tether. In some examples the distal end region of the removable securement is configured to break or otherwise fail, making it easier for the removable securement to be removed from the bone and graft by pulling the tether. In some examples, the flange portion of the removable securement is configured to be removably separated from at least a portion of the one or more prongs (posts).

As mentioned, these methods may be specifically adapted to be a method of repairing a rotator cuff. For example, a method of repairing a rotator cuff may include removing a region of a cortical layer of a humerus to form a decorticated surface; positioning a graft over the decorticated surface; temporarily securing the graft over the decorticated surface using a removeable securement, wherein temporarily securing comprises passing one or more prongs of the removeable securement through the graft and the decorticated surface; suturing a tendon over the graft and at least a portion of the removeable securement using one or more sutures; and removing the removeable securement from between the graft and the tendon, leaving the tendon sutured against the graft.

Passing one or more prongs (posts) of the removeable securement through the decorticated surface may comprise creating one or more marrow vents within the humerus that access underlying fluid marrow material, wherein the one or more prongs reside within the one or more marrow vents.

Removing the removeable securement may include removing the one or more prongs of the removeable securement from the decorticated surface, thereby causing the fluid marrow material to exit the one or more marrow vents and seep into the graft.

Removing the removeable securement may comprise removing the one or more prongs from the decorticated surface by pulling a tether attached to a flange portion of the removeable securement, wherein the one or more prongs extend from the flange. Pulling the tether may include pulling the tether in a non-perpendicular direction with respect to a long axis of the one or more prongs. Pulling the tether may cause the removeable securement to pivot such that the one or more prongs exit corresponding one or more opening within the decorticated surface.

The methods may further include forming microfractures in the decorticated surface. Such microfractures may be in addition to microfractures created by inserting the removeable securement.

Securing the graft may include anchoring the one or more tendon-securing sutures to the humerus and crossing the one or more tendon-securing sutures over the tendon.

The graft may include a first layer of demineralized bone and a second layer of mineralized bone that is continuously adjacent to the first layer, wherein positioning the graft over the decorticated surface comprises contacting the second layer with the decorticated surface. Securing the tendon against the graft may include securing the tendon against the first layer of the graft.

Suturing the tendon may include suturing the tendons of one or more of: the supraspinatus muscle, infraspinatus muscle teres minor muscle or the subscapularis muscle.

In some examples, a removable securement delivery tool comprises: a handle; and an elongate body attached to a handle, a distal end of the elongate body including a delivery portion configured to deliver a removeable securement in a bone, wherein the deliver portion includes one or more pins adapted to fit through and engage with one or more tubular posts of the removeable securement, each of the one or more pins including a sharp distal tip configured to penetrate bone and create a microfracture hole within the bone, wherein the delivery portion is adapted to release the removeable securement such that the one or more tubular posts of the removeable securement remain within the one or more microfracture holes.

The delivery portion may include an engagement surface that is adapted to engage with a flange portion of the removeable securement. The one or more pins may be configured to translate within the elongate body and relative to the engagement surface. A side of the elongate body may include a recessed channel configured to accommodate a suture attached to removeable securement. The delivery portion may include one pin adapted to fit through and engage with one tubular post of the removeable securement. The delivery portion may include two pins adapted to fit through and engage with two tubular posts of the removeable securement.

In some examples, a removeable securement adapted for use during a rotator cuff repair procedure is described. The removeable securement comprises: a flange having a substantially flat cross section, the flange including opening with a suture looped therethrough; and one or more posts extending from the flange, each of the one or more posts having a central opening extending therethrough.

Each of the one or more posts may include a tapered distal end. The flange may have a U-shaped, wherein the openings are within an apex region of the U-shaped flange. A thickness of the flange may be less than about 1 mm. Each of the one or more posts may have a length ranging from about 2 mm to 10 mm. Each of the one or more posts may have a diameter ranging from about 0.8 mm and 1.5 mm. The suture may be made of a synthetic non-absorbable material and has a diameter ranging from about 0.01 mm and 0.8 mm.

Suturing the tendon against the graft may comprise suturing with the one or more sutures. Suturing the tendon may comprise suturing the tendons of one or more of: the supraspinatus muscle, infraspinatus muscle teres minor muscle or the subscapularis muscle.

Passing the one or more sutures through the thickness of the graft may comprise passing the one or more suture through holes pre-formed through the graft. Alternatively or additionally, the grafts may permit a needle to be passed through the graft without requiring a pre-formed hole.

One innovative aspect of the subject matter described in this disclosure may be implemented as an apparatus for delivering implant material percutaneously to a patient. The apparatus may include an applicator handle, a shaft coupled to and extending distally away from the applicator handle, a needle enclosed within the shaft extending distally away from the applicator handle and protruding from a distal face of the shaft, and a cannula movably coupled to the shaft and configured to receive implant material, wherein relative motion between the shaft and the cannula causes the implant material to push out of the cannula.

In some examples, the distal face of the shaft may be configured to contact the implant material within the cannula. In some other examples, the needle may be configured to extend into the implant material. Furthermore, the needle may be configured to extend from the distal face of the shaft by an adjustable amount.

In some examples, the needle may be configured to extend into the implant material by an adjustable amount. The cannula of the apparatus may include a slot to receive the implant material. In some examples, the cannula may include one or more retention cars configured to hold the implant material within the cannula.

In some examples, the cannula may include one or more grooves configured to receive the shaft. In some other examples, the apparatus may include an extension arm configured to extend through the cannula. The extension arm may be configured to couple with the applicator handle.

Another innovative aspect of the subject matter described in this disclosure may include a method for delivering implant material into an implant region. The method may include inserting a cannula into a patient, wherein a distal end of the cannula is positioned near the implant region, and placing the implant material within the cannula, engaging a shaft of an applicator into the cannula. The method may further include extending a needle from a distal face of the shaft into the implant material and pushing the implant material out of the cannula and into the implant region.

In some examples, engaging the shaft may include contacting the implant material with a distal face of the shaft. In some other examples, pushing the implant material may include moving the shaft into the cannula.

In some examples, inserting the cannula into the patient may include inserting an obturator into the cannula. In some other examples, the method may include inserting an extension arm with the shaft through the cannula. In some cases, the extension arm may manipulate the implant material.

In some examples, the method may include withdrawing the needle and the shaft from the cannula. In some other examples, the method may include extending the needle approximately half way through the implant material.

Another innovative aspect of the subject matter described in this disclosure may include an apparatus for delivering implant material percutaneously to a patient. The apparatus may include a handle, a shaft coupled to and extending distally from the handle, and a plurality of engagement jaws configured to extend through the shaft and protrude from a distal face of the shaft and engage with implant material proximate to the distal face of the shaft.

In some examples, the handle of the apparatus may be configured to receive the plurality of engagement jaws. In some other examples, each engagement jaw may include a grip configured to provide a surface for a clinician to control the engagement jaw, an end configured to engage with the implant material, and a rod configured to couple the end to the grip. In still other examples, the plurality of the engagement jaws may be configured to be removed from the shaft.

The plurality of engagement jaws of the apparatus may include at least a first engagement jaw and a second engagement jaw. An end of the first engagement jaw may include a plurality of rods arranged in a plane to engage with the implant material and an end of the second engagement jaw may include a single rod to engage with the implant material. In some examples, the handle of the apparatus may be at least one of a cylindrical handle, a pistol-grip handle, or a t-handle.

Another innovative aspect of the subject matter described in this disclosure may include a method for delivering implant material into an implant region. The method may include inserting a cannula into a patient, where a distal end of the cannula is positioned near the implant region and placing the implant material onto first and second engagement jaws of an applicator, where the engagement jaws extend beyond a face of a shaft. The method may further include inserting, with the first and second of engagement jaws, the implant material through the cannula, removing the first engagement jaw from the implant material, and affixing the implant material to the patient.

In some examples, removing the first engagement jaw from the implant material may include moving a grip coupled to the first engagement jaw proximally away from the face of the shaft. In some examples, the shaft may be coupled to the handle and the shaft may extend distally away from the handle.

In some examples, the second engagement jaw may include a plurality of rods arranged in a plane. The second engagement jaw may provide pressure to the implant material while the implant material is being affixed to the patient.

Another innovative aspect of the subject matter described in this disclosure may include a transiently liquid-tight cannula system that includes a cannula having a proximal end and a distal end, a removable dam assembly configured to thread onto the proximal end of the cannula and resist liquid flow through the cannula, and a removable obturator configured to pass through the removable dam, extend and protrude through the distal end of the cannula.

In some cannula systems the removable obturator may be configured to detachably engage with the removable dam assembly and apply a twisting motion through the removable dam assembly to the cannula.

Any of the cannulas described herein may include threads configured to draw the cannula into a patient when a twisting motion is applied to the cannula. Furthermore, any of the cannulas described herein may include a threaded section configured to engage with and attach to the removable dam assembly.

In some cannula systems, the removable dam assembly may include a conforming gasket configured to resist liquid flow and an engagement surface configured to detachably engage with the removable obturator. The conforming gasket may be configured to allow the removable obturator to pass therethrough.

In various cannula systems, the removable obturator may include a handle and the engagement surface of the removable obturator may be distal with respect to the handle. A distal end of the removable obturator may be conically shaped.

Another innovative aspect of the subject matter described in this disclosure may include a method that includes inserting a cannula system into a surgical portal, where the cannula system includes a cannula having a proximal end and a distal end, a removable dam assembly configured to thread onto the proximal end of the cannula and resist liquid flow through the cannula, and an obturator configured to pass through the removable dam assembly, extend and protrude through the distal end of the cannula. The method may further include removing the obturator, inserting implant material through the cannula into an implantation area, and affixing the implant material to a patient.

In some examples, the method may further include removing the removable dam assembly from the cannula prior to inserting the implant material through the cannula. Furthermore, the method may include inserting the implant material through the removable dam assembly and re-attaching the removable dam assembly to the cannula.

Various removable dam assemblies may include an engagement surface configured to detachably engage with the engagement surface of the obturator. In some examples of the method, inserting the cannula may include twisting the obturator to cause the cannula to be drawn through the surgical portal. In some examples, the cannula system may be configured to resist liquid flow after the obturator is removed.

Another innovative aspect of the subject matter described in this disclosure may include another transiently liquid-tight cannula system that includes a splitable cannula having a proximal end and a distal end and a removable obturator configured to form a liquid-tight scal with the splitable cannula, wherein the proximal end of the splitable cannula is configured to receive the removable obturator.

In some aspects, the cannula system may include a removable fixation configured to temporarily hold together the splitable cannula.

In some examples, the distal end of the splitable cannula is configured to be inserted into an implantable access portal. In some further examples, the splitable cannula may be configured to receive an applicator and implant material while inserted into the implantable access portal.

In some other examples, the splitable cannula may be configured to separate into two or more pieces to facilitate removal from an applicator while the applicator is inserted through the splitable cannula and an implantable access portal.

In still other examples, the splitable cannula may include at least a first member and a second member and the first member and the second member include locking features to detachably couple the first member to the second member.

Another innovative aspect of the subject matter described in this disclosure may include a method that includes inserting a splitable cannula system into an implantable access portal, where the splitable cannula system includes a splitable cannula having a proximal end and a distal end, a fixation device configured to temporarily hold the splitable cannula together, and an obturator configured to pass through the splitable cannula. The method may further include removing the obturator from the splitable cannula system, inserting a delivery system holding implant material through the splitable cannula and the implantable access portal into an implantation area, and removing the splitable cannula from the implantable portal.

In some examples, removing the splitable cannula may include separating the splitable cannula into two or more sections while the delivery system remains within the implantable access portal. In some other examples, removing the splitable cannula may include overcoming the fixation device to separate the splitable cannula into two or more sections.

In some examples, removing the splitable cannula may include grasping the distal end of the splitable cannula and moving the splitable cannula in a proximal direction.

In some examples, the disclosed methods may include affixing the implant material to the implantation area.

All of the methods and apparatuses described herein, in any combination, are herein contemplated and can be used to achieve the benefits as described herein.

### BRIEF DESCRIPTION OF THE DRAWINGS

A better understanding of the features and advantages of the methods and apparatuses described herein will be obtained by reference to the following detailed description that sets forth illustrative embodiments, and the accompanying drawings of which:

FIG. 1 illustrates an example of a bone graft material (e.g., scaffold) having a layer of demineralized and a layer of mineralized bone.

FIGS. 2(1)-2(10) illustrate one example method of implanting/inserting a bone graft to repair a connective (e.g., tendon) tissue.

FIGS. 3A-3B schematically illustrate removeable securements adapted to temporarily secure a bone graft to an attachment region of a bone.

FIGS. 3C-3E schematically illustrate methods of using a removable securement to repair an enthesis as described herein.

FIGS. 4A-4D illustrate an exemplary removeable securement.

FIGS. 4E-4G illustrate an example of a removeable securement tool (removable securement insertion tool) for inserting a removeable securement thorough a graft and into bone.

FIG. 5A illustrates an example of a removeable securement.

FIGS. 5B-5D show an example of a removable securement delivery tool for inserting a removeable securement (such as that shown in FIG. 5A) through a graft and into bone.

FIGS. 6A-6D illustrate exemplary features that may be incorporated into any of the removable securement delivery tools described herein.

FIGS. 7A-7G illustrate an exemplary method for repairing an enthesis using the methods and one example of a removable securement as described herein. Not to scale; the removeable securement is shown enlarged to illustrate the method.

FIG. 8 illustrates an example of a tendon secured to a bone using one or more sutures (the graft may be secured between the tendon and the bone). The suture includes knots.

FIG. 9 illustrates another example of a suture securing a tendon to bone, where the suture is knot-less.

FIGS. 10A-10C illustrate preparing the graft and bone region before preforming any of the methods described herein.

FIG. 11 is a simplified diagram of an implant delivery system, in accordance with some examples.

FIG. 12 is a simplified diagram of an applicator handle, in accordance with some examples.

FIG. 13 is a simplified diagram of a cannula, in accordance with some examples.

FIG. 14 is another simplified diagram of a cannula, in accordance with some examples.

FIG. 15A shows a proximal end view of a cannula, in accordance with some examples.

FIG. 15B shows another proximal end view of the cannula, in accordance with some examples.

FIG. 15C shows an example overhead view of the cannula.

FIG. 16 shows a proximal end view of a cannula and a shaft.

FIG. 17 shows a distal end view of the end of a shaft, in accordance with some examples.

FIG. 18 is a flowchart showing an example method for delivering implant material into an implant region, in accordance with some examples.

FIG. 19 is a simplified diagram of another implant delivery system, in accordance with some examples.

FIG. 20 shows jaws, in accordance with some examples.

FIG. 21 is a simplified diagram of another example implant delivery system, in accordance with some examples.

FIG. 22 is a flowchart showing an example method for delivering implant material into an implant region, in accordance with some examples.

FIG. 23 is a simplified diagram of another example implant delivery system, in accordance with some examples.

FIG. 24 is a simplified diagram of another example implant delivery system.

FIG. 25 is a simplified diagram of another example implant delivery system.

FIG. 26 is a cross section view of an implant delivery system.

FIG. 27A shows a distal view of engagement jaws.

FIG. 27B shows a cross section view of a shaft.

FIG. 28 shows a transiently liquid-tight cannula system.

FIG. 29 shows an exploded view a cannula system.

FIGS. 30A-30C show additional images of the cannula system of FIG. 29.

FIGS. 31A-31G illustrate different configurations of the cannula system of FIG. 29 while being used to deliver implant material to an implant region.

FIG. 32 is a flowchart showing an example method for delivering implant material into an implant region, in accordance with some examples.

FIG. 33 shows a splitable cannula system.

FIG. 34 shows an exploded view a splitable cannula system.

FIGS. 35A-35G illustrate different configurations of the splitable cannula system of FIG. 34 while being used to deliver implant material to an implant region.

FIG. 36. Is a flowchart showing an example method for delivering implant material into an implant region, in accordance with some examples.

FIGS. 37A-37D illustrates an example of an implant delivery apparatus as described herein. FIG. 37A shows a perspective view of the implant delivery apparatus. FIG. 37B shows a longitudinal section through the implant delivery apparatus of FIG. 37A. FIG. 37B shows an enlarged view of the handle region of the implant delivery apparatus of FIG. 37B. FIG. 37D shows an exploded view of the apparatus of FIGS. 37A-37C.

### DETAILED DESCRIPTION

Described herein are methods, devices and systems for delivering and securing a graft implant to tissue (e.g., bone) during a surgical procedure. The methods and tools are well suited for delivering and implanting connective tissue-to-bone interface grafts that are surgically implanted onto bone to allow ingrowth of both bone and connective tissue. These methods and tools particularly well adapted for implantation and securement as part of a minimally invasive, e.g., arthroscopic, laparoscopic, surgery.

Example connective tissue-to-bone interface grafts may include a demineralized porous structure that can act as a scaffold for cellular attachment and proliferation. In some examples, the graft has a layered structure including at least two layers, such as a demineralized layer and a mineralized layer. Examples of such grafts (e.g., scaffolds) are described in U.S. patent application Ser. No. 17/015,043, which is incorporated herein by reference in its entirety.

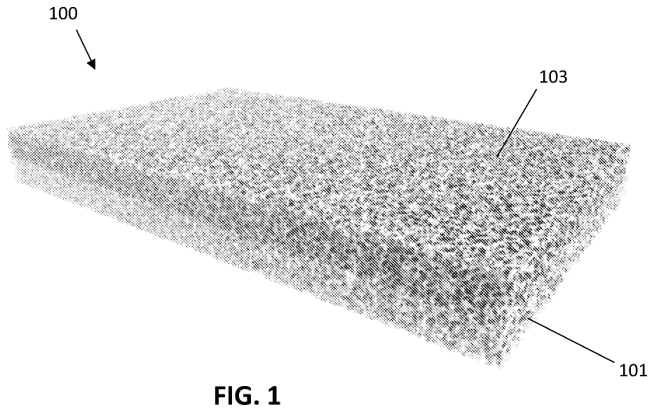

FIG. 1 shows one example of a tissue-to-bone interface graft (e.g., scaffold) 100 having a layered structure. The graft includes a first, demineralized layer 101 and a second, mineralized layer 103. In some cases, the dimensions of the mineralized and demineralized layers, and in particular, their ratios, may be selected within a desired range to optimize both the case of using them in the particular surgical procedures, as well as for stability and in-growth of connective tissue and/or bone. In FIG. 1 the mineralized and demineralized portions are both porous, although they may have different porosities. For example, the porosity of the mineralized side of the implant may be greater than (or the same as) the porosity of the demineralized side, and may be configured or selected to permit the fluid pressure from the humerus to drive bone marrow into the graft (e.g., into the cancellous side of the graft). In some examples the implant may be configured to limit the migration of the bone marrow into the demineralized side, e.g., by limiting the porosity and/or by treatments to limit migration (including treatments to reduce the side, modify the surface properties, provide a barrier to migration, etc.). In some examples the graft may be configured to permit migration of the bone marrow into the demineralized side.

The graft may be shaped and sized so that it may be inserted or implanted into the body, as will be described in greater detail below, as well as providing optimal attachment to the bone and connective tissue. In the example shown in FIG. 1, the biocompatible bone graft has a rectangular or approximately rectangular (e.g., rectanguloid) shape. In some cases, graft may have one or more rounded edges. In some examples, the graft has a structure dimensioned somewhat like a stick of gum, e.g., having a flat body that is longer than it is wide, and substantially thinner than it is wide or long.

In some examples, the grafts may comprise a collagen-based porous network capable of guiding tissue differentiation that can be used to regrow damaged soft tissues (e.g., connective tissue). The relatively high porosity, e.g., of either or both the demineralized and mineralized material may allow host integration, regeneration of relatively large sections of tissue, and vascularization. The collagen-based porous structure may allow binding of a variety of factors to the trabecular (pores) within the graft formed of bone. Additional materials, such as hydrogels or extracellular matrix material, and/or a variety of biological components and therapeutic compounds may be integrated within the graft. The grafts may contain collagen trabecular that may allow the structure to maintain a pre-defined shape and maintain nutrient transport. Thus, the grafts may desirably provide mechanical integrity, nutrient transport during tissue regeneration, differentiation of well-defined cell populations, vascularization.

As used herein, the term “biocompatible” may refer to any material having a relatively low risk of provoking an adverse response when introduced in a mammal, in particular a human patient. For example, a suitable biocompatible material when introduced into a human patient has relatively low immunogenicity and toxicity. The term “demineralized” may refer to bone from which a substantial portion of minerals natively associated with the bone minerals have been removed. The term “demineralized bone” is intended to refer to any bone, including cortical and/or cancellous bone, from any source including autologous, allogeneic and/or xenogeneic bone, that has been demineralized to contain, in certain examples, less than about 8 wt % residual calcium (e.g., less than about 7 wt % residual calcium, less than about 6 wt % residual calcium, less than about 5 wt % residual calcium, less than about 4 wt % residual calcium, less than about 3 wt % residual calcium, less than about 2 wt % residual calcium, or less than about 1 wt % residual calcium, etc.).

A graft may be referred to as “substantially free of mineralized bone” when all of the bone within the graft has been exposed to demineralizing conditions and is at least partially demineralized. Graft that are substantially free of mineralized bone are structurally and functionally distinct from grafts made from bone that has been masked prior to demineralization (see, e.g., U.S. Published Application 20110066241).

In certain examples, the surface structure of the graft may be modified to provide texture, roughness and/or three-dimensional unevenness to the graft. The surface roughness of the graft may be altered by chemical etching or by physical etching. The grafts described herein may also be used as delivery devices for therapeutics, wherein the therapeutic comprises the minced tissue, which may include a combination of cells, extracellular matrix and/or inherent growth factors. The graft may thus permit hormones and proteins to be released into the surrounding environment.

The graft may be useful in treating injuries involving interfaces within connective tissues. The major applications include repair of ligaments, tendons, and cartilage. Ligaments are dense bands of connective tissue composed primarily of type I collagen that connect bones to other bones. Ligaments function as motion guides and joint motion restrictors. At all articulating joints (neck, spine, shoulder, elbow, wrist, hip, knee, ankle) in the body, these tissues are placed under constant dynamic loading. An injury known as a sprain results when the ligaments are stretched, and in some cases, stretched severely enough to be torn. While in some cases, ligament tears can heal on their own, other cases show a lack of inherent healing capacity. If left untreated or if treated improperly, ligament tears can lead to chronic disability including arthritis at the affected joint. Tendons, like ligaments, are dense collagenous tissues found at every articulating joint in the body. Tendons, however, connect muscles to bone, allowing the force produced by the muscles to be translated into motion. When overloaded, tendons are at risk for tearing and in some cases require surgical replacement to return joint motion and prevent muscle atrophy.

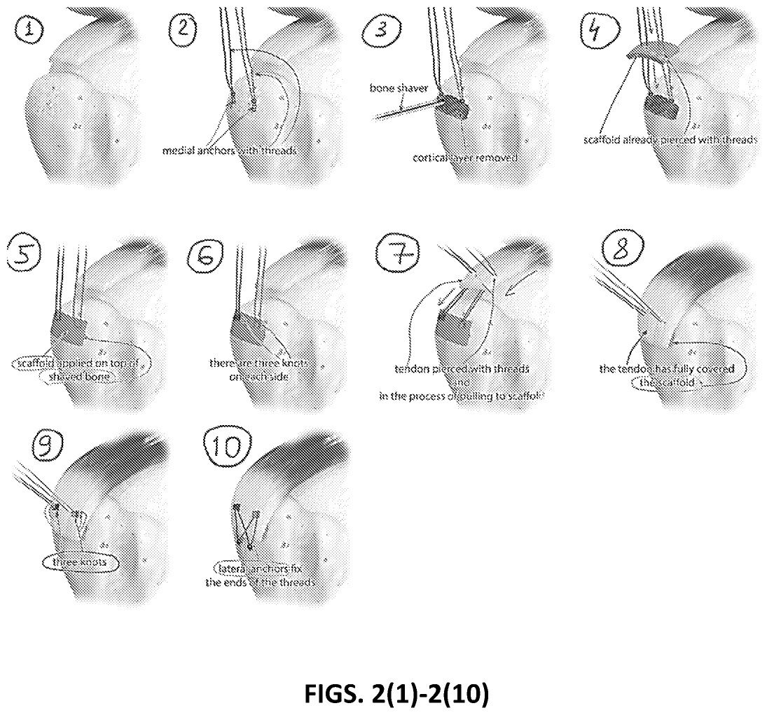

According to some examples, the graft is implanted and held in place at an implant site by an anchoring and suturing system. FIGS. 2(1)-2(10) illustrate one exemplary method of implanting a graft with an anchoring and suturing system, in this case, to repair a rotator cuff. In FIG. 2(1), the tissue to be repaired may be prepared, e.g., by removing unwanted tissue and/or debriding. In FIG. 2(2), the boney region to which the graft will be anchored is prepared, by applying one or more anchors (e.g., medial anchors) including sutures (“threads”). A bone shaver may be used to cut away the region into which the graft will be applied, as shown in FIG. 2(3). The outer cortical layer of bone may be removed to reveal the vascularized, growth factor rich cancellous bone below (e.g., decortication footprint). Such removal of outer bone may be referred to as decortication. The mineralized side of the graft may be placed down onto the exposed inner bone region, so that the marrow may be received into the trabecula of the mineralized bone helping to vascularize the graft to the native bone tissue. In FIG. 2(4), the graft (which may be preloaded on the sutures, e.g., via one or more suture channels may be slid down through a cannula (not shown) and into the bone. In FIG. 2(5), the graft is applied onto the prepared bone and may be anchored, via the suture, in place, as shown in FIG. 2(6). In FIG. 2(7), the tendon to be repaired may be sutured with the same sutures used to anchor the graft (or coupled to the graft anchors) and may be pulled down onto the graft outer surface (e.g., the demineralized layer of the graft). FIG. 2(8) shows the tendon fully covering the graft, and in FIG. 2(9) and FIG. 2(10) the sutures may be used to anchor and secure the tendon to the graft. In some examples additional material may be used to secure the tendon to the graft. In some examples, no additional securement or augmentation is not necessary. The method illustrated in FIGS. 2(1)-2(10) uses a suture to hold the graft in place, and the suture (e.g., knots) remain between the graft and the tendon. In contrast, described herein are methods that may secure the graft in position without leaving any material (including suture material) between the tending and the graft or the graft and the bone.

It may be beneficial to complete the procedure without leaving any attachment materials (surgical materials) between the graft and the bone and/or between the graft and the tendon. In particular, it may be beneficial to allow the graft to be held between the tendon and the bone only by the tendon attachment. This may enhance healing.

For example, the graft may be temporarily secured to the bone by a temporary removeable securement prior to and/or during suture and securement of the tendon or ligament to the bone. The temporary removeable securement may have a form factor and size that prevent it from interfering with suturing of the tendon (e.g., double arrow cuff repair). Once the tendon or ligament is sutured and secured to the bone, the secured tendon or ligament can provide a compression force on the graft that is sufficient to prevent the graft from migration. Thus, the temporary removeable securement can be extracorporeally removed, thereby minimizing concerns of foreign body response or tissue damage resulting from a long-term implanted component within or near the biological graft.

In some examples, the temporary removeable securement includes one or more pins or needles (e.g., 18-gauge needles) that are percutaneously deployed through the graft and into the decorticated cancellous bone. The lateral anchors can then be placed, the percutaneous needles removed, and the tendon or ligament surgically sutured and repaired as usual.

In some examples, the temporary removeable securement is any suitable atraumatic arthroscopic tool. The atraumatic arthroscopic tool can be manipulated to hold the graft in place over the graft site while medial anchors are passed through the graft. Then the tendon or ligament can be surgically sutured and repaired as usual.

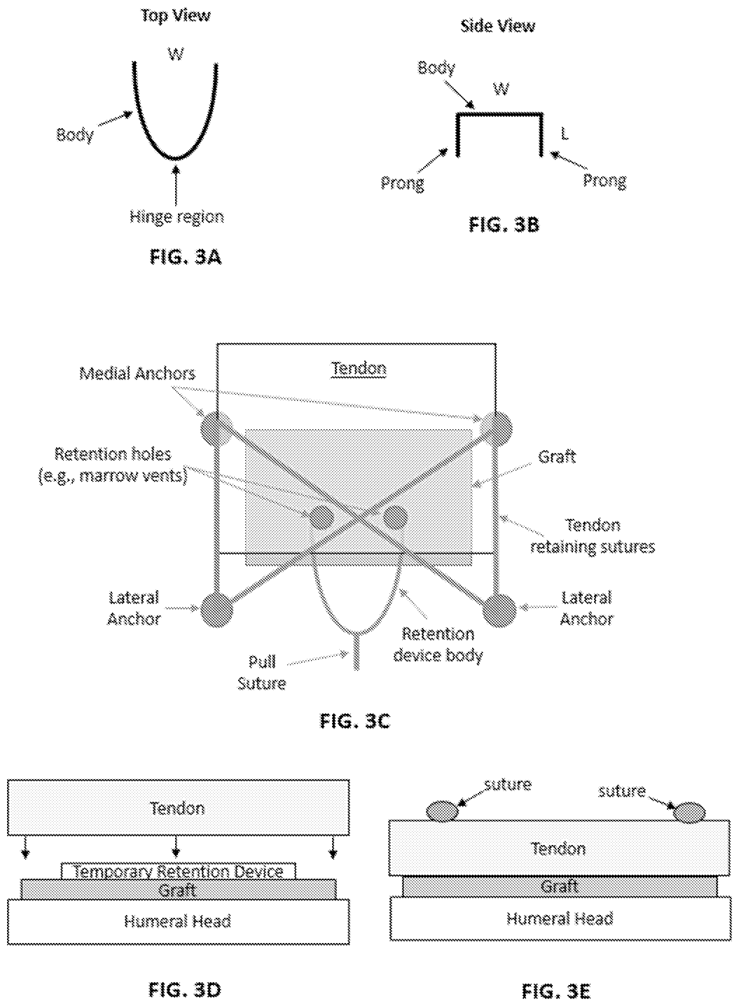

In some examples, the temporary removeable securement is configured to pass through the graft and through one or more holes formed in the underlying bone. FIGS. 3A-3E illustrate an example of such a removeable securement system, including a removable securement (FIGS. 3A-3B) and a method of using the system for removably inserting the removable securement. In this example, the removeable securement may include a body (also referred to herein as a flange), as shown in the top view of FIG. 3A, and two prongs (also referred to herein as posts) that extend from the body, as shown in the side view FIG. 3B. As shown, the body has U-shape and a flat (e.g., substantially planar) cross-section. A central region at an apex of the U-shaped body (which may be referred to as a hinge region) can be attached to a suture (not shown) that can be pulled to remove the device. The length L of the prongs may be sufficiently long to penetrate through the graft and into the bone but short enough to allow for easy removal of the device. In some examples, the length L of the prongs is about 2.5 mm or less. In other examples, the length L of the prongs is from about 2 mm and 10 mm. The length may be chosen based on the thickness of the graft (e.g., the length may be between 0.4 mm and 5 mm, between 0.5 mm and 3 mm, etc. the thickness of the graft). The width W of the body may be wide enough to sufficiently retain the graft in place but narrow enough to allow for easy removal of the device. In some examples, the width W of the body is about 5 mm or less. The width of the body may also allow for easy removal of the device. In some examples, the width of the body is about 15 mm.

The material of the removeable securement may vary. In some examples, the temporary removeable securement is made of a shape memory material (e.g., Nitinol). In some examples, the removeable securement may be configured to create an outward force at the prongs that retains the device within retention holes within the bone. In one example, the removeable securement is heat set such that the prongs have a width greater than the body (e.g., >5 mm). When released from a delivery system, the removeable securement device may attempt to revert to the wider configuration, thereby placing an outward force on the anterior face of an anterior retention hole and outward force on the posterior face of a posterior retention hole.

Alternatively or additionally, the removeable securement may include a polymeric material. In some examples, the polymeric material may be configured to collapse and expand to provide an interference fit within the retention holes within the bone. For example, the device may include an expandable/collapsible balloon or a thin-walled tube. In some cases, the device includes a polymeric clip that is inserted on an angle to impinge with walls in the retention hole.

FIG. 3C shows an example placement of the removeable securement with respect to a graft, a tendon and suture anchors. The prongs of the removeable securement can pass through the graft and within retention holes formed within the underlying bone. In some examples, the retention holes may be marrow vents and may be formed using a puncture tool (e.g., removable securement delivery tool or microfracture tool), as described herein. The flat body of the removeable securement can enable the body to fit between the graft and an overlying tendon without interfering with the suturing of the tendon to the underlying bone. In some cases, the removeable securement may be positioned midline with respect to the medial anchors and lateral anchors to prevent entrapment under the straight medial to lateral sutures. The hinge region of the body may be positioned laterally outside of the graft and/or tendon.

Once the tendon is sutured and at least partially secured, the retaining device is no longer needed and can be removed. This can be done by pulling a tether attached to the removeable securement body in a non-parallel direction with respect to the flat removeable securement body (e.g., down), as accommodated by the curvature of the bone and surrounding anatomical structure. This can cause the device to pivot/rotate to an extent that the prongs are released from the retention holes. Upon further pulling of the tether, the removeable securement can be removed from between the graft and the tendon and removed from the patient's body. It should be noted that other removal “handles” could be placed indwelling instead of, or in addition to, the tether. In one example, a short tether (e.g., <1 inch) or a handle that is amenable to being firmly gripped by arthroscopic graspers may be implemented. In some cases the removable securement device may be removed when the tendon is partially secured (leaving room for removal); the tending may be completely fixed down, securing the graft, after removing the removable securement device. In some examples, the removable securement device may be removed after the tendon is secured.

FIGS. 3D-3E schematically illustrates cross section view showing a location of the removeable securement with respect to the tendon, graft and bone (e.g., humoral head) during the procedure (FIG. 3D) and after the procedure (FIG. 3E). Since at least a portion of the removeable securement is positioned between the tendon and the graft, the device should be nominally thin so as not to interfere with the graft or tendon securement. The temporary removeable securement should also be atraumatic with regard to the soft tissue and suture of the repair during removal of the removeable securement.

As described above, the prongs of the removeable securement may be retained within retention holes formed within the bone. In some cases, the retention holes may be sufficiently deep to reach the marrow material and blood. Once the removeable securement is removed, the marrow material can seep into the repair site, including the graft, providing a therapeutic benefit and promote healing by increasing native growth factors and stem cells at the repair site. In this way, the retention holes may not only retain the removeable securement but also serve to improve the surgical outcome. Such marrow venting may be used in place of a separate marrow venting procedure (e.g., microfracture procedure), which could add to the procedure costs. Alternatively, a separate marrow venting procedure (e.g., microfracture procedure) may be implemented in addition to forming the retention holes for the removeable securement.

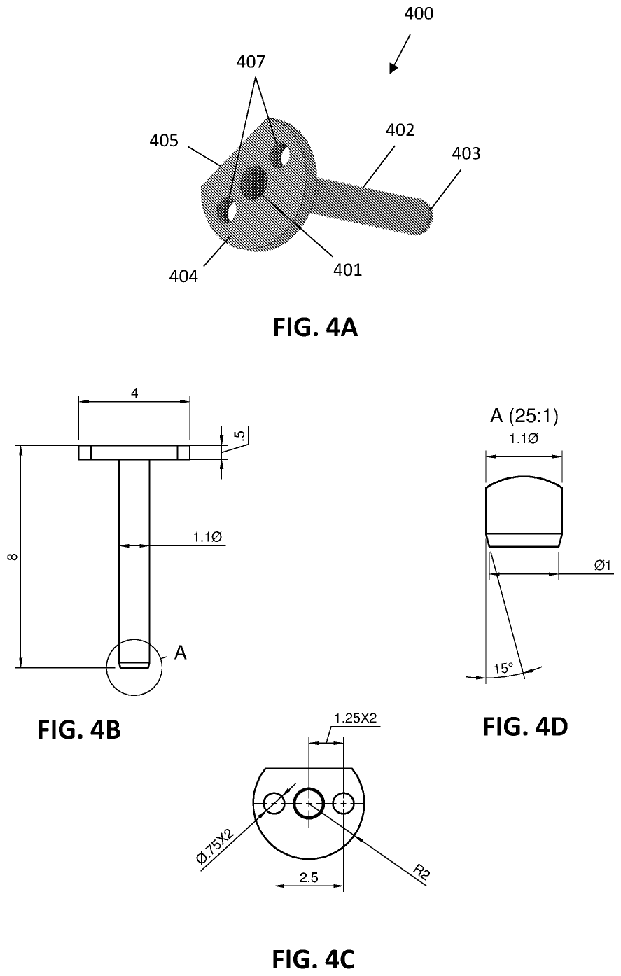

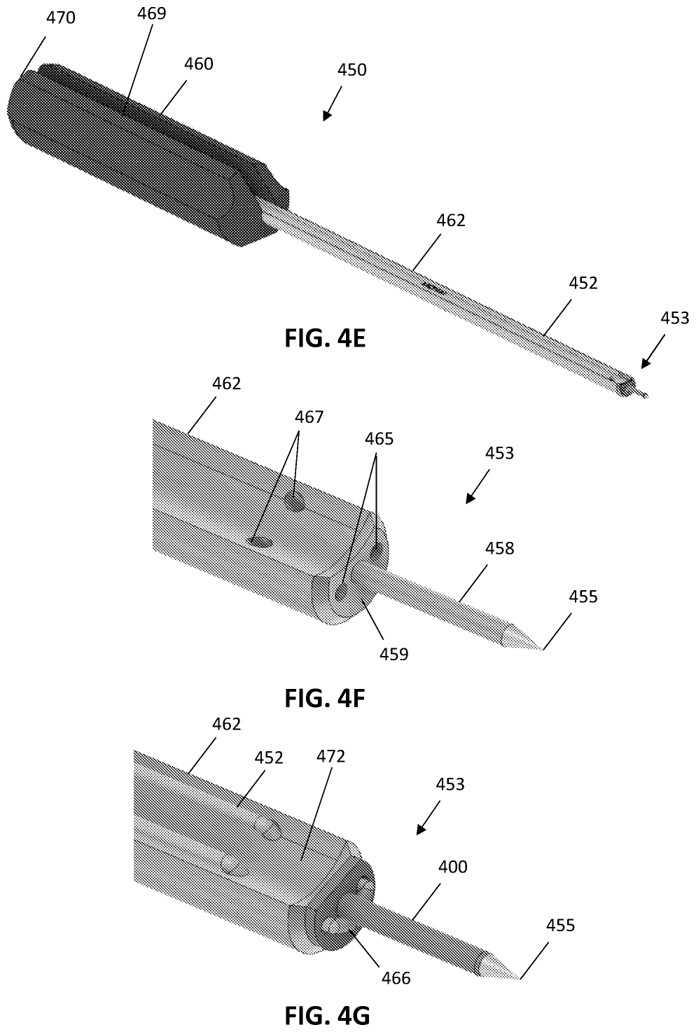

FIGS. 4A-4G illustrate another exemplary temporary removeable securement 400 and corresponding removeable securement insertion tool 450. As shown in FIG. 4A, in this example, the device 400 includes a flange 404 with one prong 402 (also referred to as a post) extending therefrom. The flange 404 has a flat cross section that is nominally thin to minimized distance between the graft and tendon, as described herein. The flange includes openings 407 arranged to accommodate a suture. The prong 402 includes a central opening 401 that is configured to accommodate insertion of a delivery portion of the removeable securement insertion tool. Thus, the prong 402 corresponds to a tubular component of the device 400. In some cases, a distal end 403 of the prong 402 may be tapered to facilitate insertion within the bone. The flange 404 has a cut away portion 405, which may correspond to a flat edge of the flange 404. The cut away portion 405 may allow for easier removal of the device 400 once the tendon is secured. In addition, the cut away portion 405 may allow the suture to pass up around the device 400.

The removeable securement 400 may be made of any of a number of materials. In some examples, the device 400 is made of a biocompatible polymer and/or metal material. In some cases, the device 400 is made of a shape memory alloy (e.g., nitinol).

FIGS. 4B-4D illustrate example dimensions of the device 400 (e.g., in mm). Note that the dimensions shown are only exemplary and that the dimensions of the device 400 may vary. In some examples, the prong 402 has a length ranging from about 4 mm and 8 mm. In some examples, the diameter of the prong 402 ranges from about 0.8 mm and 1.5 mm. In some examples, a thickness of the flange 404 may range from about 0.25 mm and 1 mm. In some examples, the thickness of the flange 404 is based on a predicted height above the bone set by the maximum height of the compressed graft (e.g., 3 mm or less above the bone).

FIGS. 4E-4G show an exemplary removeable securement insertion tool 450 that is configured to create a marrow vent (e.g., microfracture) within a bone and also to deliver the temporary removeable securement 400 within the bone. As shown in FIG. 4E, the removeable securement insertion tool 450 includes an elongate body 462 having a distal end configured to couple with the temporary removeable securement 400. A handle 460 is configured for a user to manipulate the position of the device 400 and to apply an appropriate force to create the marrow vent. In some cases, the proximal end 470 of the handle 460 may be configured for accepting a force (e.g., pounding force). The handle 460 may include a channel 469 to accommodate the elongate body 462 and suture 462 loaded thereon.

FIG. 4F shows a closeup view of a delivery portion 453 at the distal end of the elongate body 452. A pin portion 458 is configured to be inserted within the central opening 401 of the removeable securement 400. The pin 458 has a pointed distal tip 455 that is configured to pierce through tissue (e.g., bone). The delivery portion 453 also includes a flat engagement surface 459 that is configured to engage with a surface (e.g., top surface that will engage with the tendon) of the flange 404 of the removeable securement 400. The delivery portion 453 further includes first openings 465 that provide access to a channel within the delivery portion 453 and that exits second openings 467 along a side of the elongate body 462.

FIG. 4G shows the temporary removeable securement 400 loaded onto the delivery portion 453. As shown, the distal end of the pin 458 can extend past the distal end of the removeable securement 400 to expose the pointed distal tip 455. A suture 452 is threaded through the openings 465/467 such that the suture 452 includes a loop 466 at the underside of the flange 404 of the removeable securement 400. The suture 452 may travel along a recessed channel 472 that runs along a side of the elongate body 462. A releasing mechanism of the tool 450 may be configured to release (e.g., push) the removeable securement 400 from the delivery portion 453 of the tool 450. In one example, the pin 458 is adapted to translate within the elongate body 452 and relative to the engagement surface 459. For example, the pin 458 can be retracted proximally while the engagement surface 459 retains the removeable securement 400 within the marrow vent, then the elongate body 462 can be retracted proximally leaving the removeable securement 400 within the marrow vent. In other examples, the elongate body 462 may be configured to provide an ejection force against the removeable securement 400 so that the elongate body 452 can be retracted proximally while leaving the removeable securement 400 within the marrow vent.

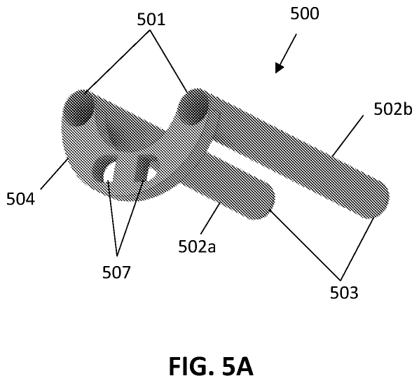

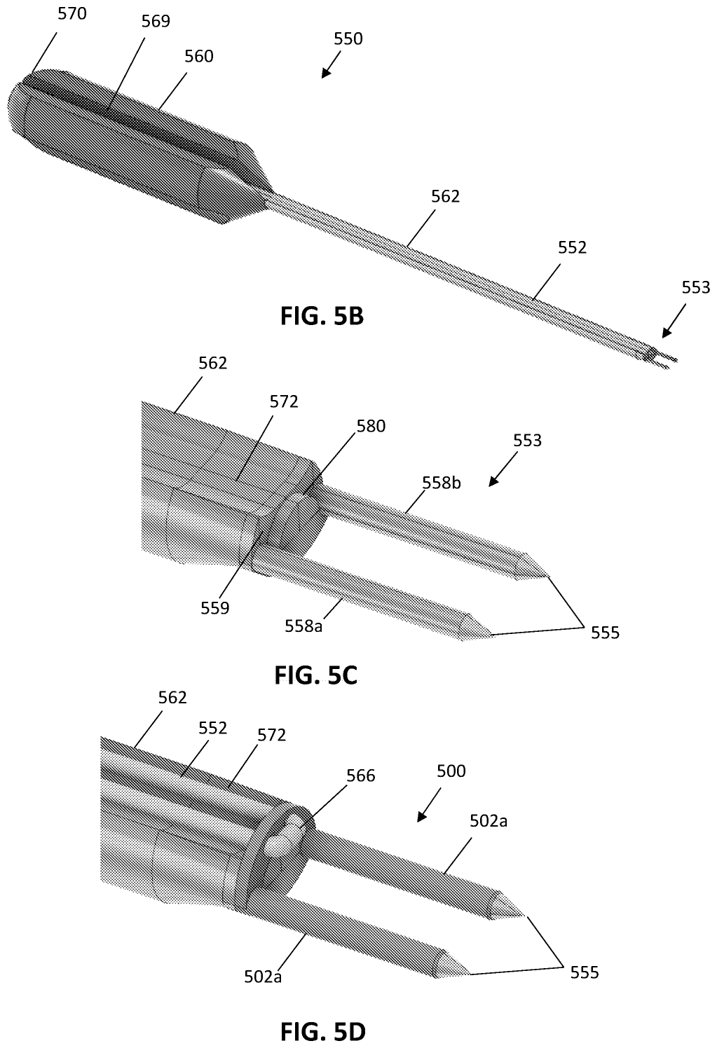

FIGS. 5A-5D illustrate another exemplary temporary removeable securement 500 and corresponding removeable securement insertion tool 550. As shown in FIG. 5A, in this example, the device 500 includes a flange 504 with two prongs 502a and 502b extending therefrom. The flange 504 has a flat cross section that is nominally thin to minimize a distance between the graft and tendon, as described herein. The flange 504 has a U-shape with the suture openings 507 located near an apex of the U-shaped flange 504 to allow for easier removal of the device 500 once the tendon is secured. The prongs 502a and 502b each includes a central opening 501 that is configured to accommodate insertion of a delivery portion of the removeable securement insertion tool. Thus, the prongs 502a and 502b correspond to tubular components of the device 500. In some cases, distal ends 503 of the prongs 502a and 502b may be tapered to facilitate insertion within the bone.

The dimensions of the device 500 may vary. In some examples, the prongs 502a and 502b have lengths ranging from about 4 mm and 8 mm. In some examples, the diameter of the prongs 502a and 502b range from about 0.8 mm and 1.5 mm. In some examples, a thickness of the flange 504 may range from about 0.25 mm and 1 mm. In some examples, the thickness of the flange 504 is based on a predicted height above the bone set by the maximum height of the compressed graft (e.g., 3 mm or less above the bone).

Similar to device 400, the removeable securement 500 may be made of any of a number of materials. In some examples, the device 500 is made of a biocompatible polymer and/or metal material. In some cases, the device 500 is made of a shape memory alloy (e.g., nitinol).

FIGS. 5B-5D show an exemplary removeable securement insertion tool 550 that is configured to create a marrow vent (e.g., microfracture) within a bone and to deliver the temporary removeable securement 500. As shown in FIG. 5B, the removeable securement insertion tool 550 includes an elongate body 562 having a distal end configured to couple with the temporary removeable securement 500. A handle 560 is configured for a user to manipulate the position of the device 500 and to apply an appropriate force to create the marrow vent. A proximal end 570 of the handle 560 may be configured for accepting a force (e.g., pounding force). The handle 560 may include a channel 569 to accommodate the elongate body 562 and a suture 562 loaded thereon.

FIG. 5C shows a closeup view of the distal end having a delivery portion 553 of the elongate body 552. Two pin portions 558a and 558b are configured to be inserted within the openings of the corresponding prongs 502a and 502b of the temporary removeable securement 500. The pins 558 have pointed distal tips 555 that are configured to pierce through tissue (e.g., bone). The delivery portion 553 also includes a flat engagement surface 559 that is configured to engage with a surface (e.g., top surface that will engage with the tendon) of the flange 504 of the removeable securement 500. The flat engagement surface 559 may be defined by a curved edge 580, which is configured to engage with, and have a corresponding shape of, an inner edge of the U-shaped flange 540 of the removeable securement 500.

FIG. 5D shows the temporary removeable securement 500 loaded onto the delivery portion 553 of the tool 550. As shown, the distal ends of the pins 558a and 558b can extend past the distal end of the removeable securement 500 to expose the pointed distal tips 555. A suture 552 is threaded through the suture openings of the removeable securement 500 such that the suture 552 includes a loop 566 at the underside of the flange 504 of the removeable securement 500. The suture 552 may travel along a recessed channel 572 that runs along a side of the elongate body 562. A releasing mechanism of the tool 550 may be configured to release (e.g., push) the removeable securement 500 from the delivery portion 553 of the tool 550. In one example, the pins 558a and 558b are adapted to translate within the elongate body 552 and relative to the engagement surface 559. For example, the pins 558a and 558b can be retracted proximally while the engagement surface 559 retains the prongs 502a and 502b within respective marrow vents, then the elongate body 562 can be retracted proximally leaving the prongs 502a and 502b within the marrow vent. In other examples, the elongate body 562 may be configured to provide an ejection force against the removeable securement 500 so that the elongate body 552 can be retracted proximally while leaving the prongs 502a and 502b within corresponding marrow vents.

Any of the removeable securement insertion tools may include various features to facilitate delivery of the removeable securement to the delivery site and/or create sufficiently deep holes within the bone to access bone marrow material. In some examples, the removeable securement insertion tool includes an adjustable head and cannula (e.g., 10 mm cannula) with a pushability within a certain distance (e.g., 9 mm). In some cases, the removeable securement insertion tool includes a drive mechanism similar to a stapler or riveter to drive the removeable securement into the bone, which can allow for easy insertion and removal of the tool. In some cases, the removeable securement insertion tool can allow for deployment in non-decorticated cortical bone.

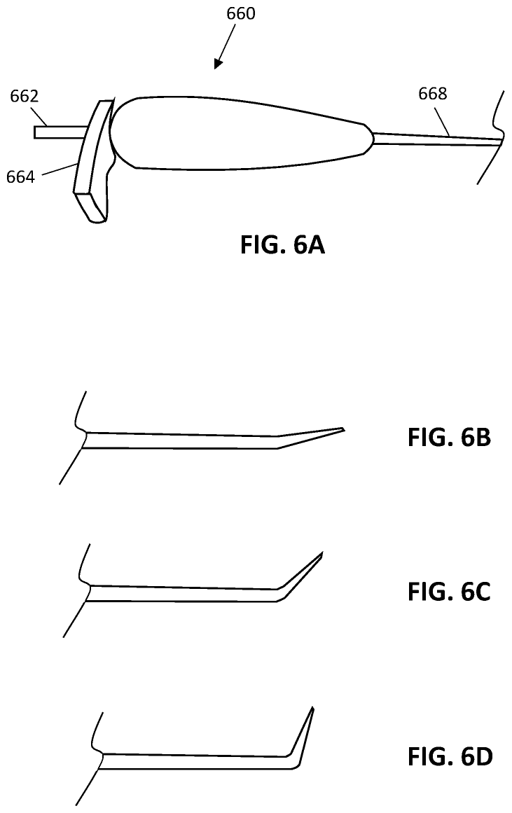

FIGS. 6A-6D illustrate exemplary features that may be incorporated into any of the removeable securement insertion tools described herein. FIG. 6A shows a portion of an exemplary tool having a handle 660 having a proximal end of a drive pin 662 and drive pin release lever 664. To create a microfracture within the bone, the surgeon may use a mallet to hit the proximal end of a drive pin 662, which translates distally and provides a driving force toward the distal end of the elongate body 668 to create a microfracture within the bone. The distal end of the elongate body 668 can include a delivery portion (e.g., 453 or 553) that is configured to drive a temporary removeable securement (e.g., 400 or 500) into the bone. After a microstructure is formed, the surgeon can use the drive pin release lever 664 to move the drive pin 662 back proximally to retract the elongate body 668 out of the microfracture hole, leaving the temporary removeable securement within the bone.

The elongate body of the removeable securement insertion tool may have one or more bends to facilitate access to the puncture sites of the bones. FIGS. 6B-6D show distal portions of various exemplary elongate bodies having different degrees of bend.

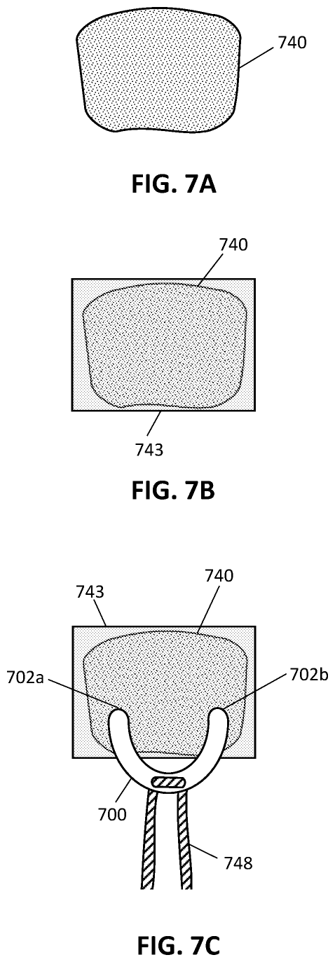

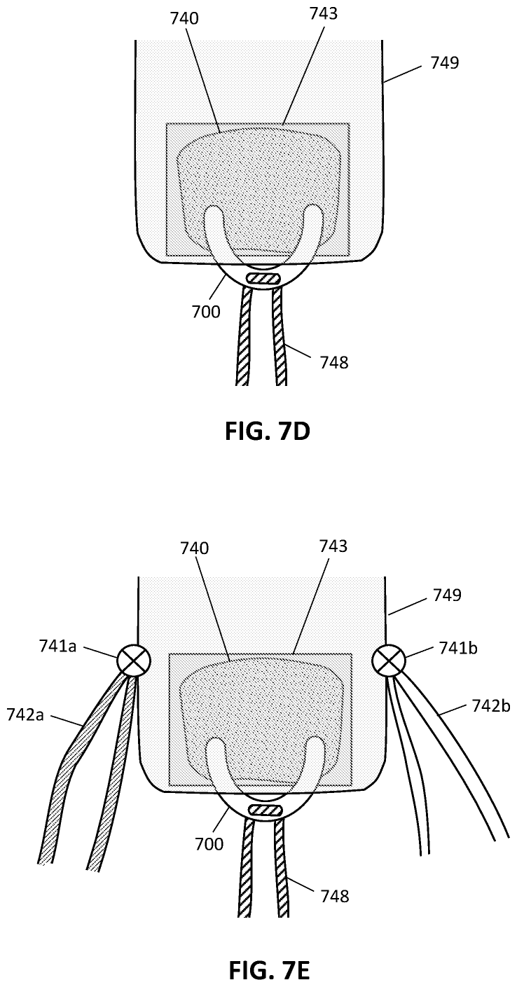

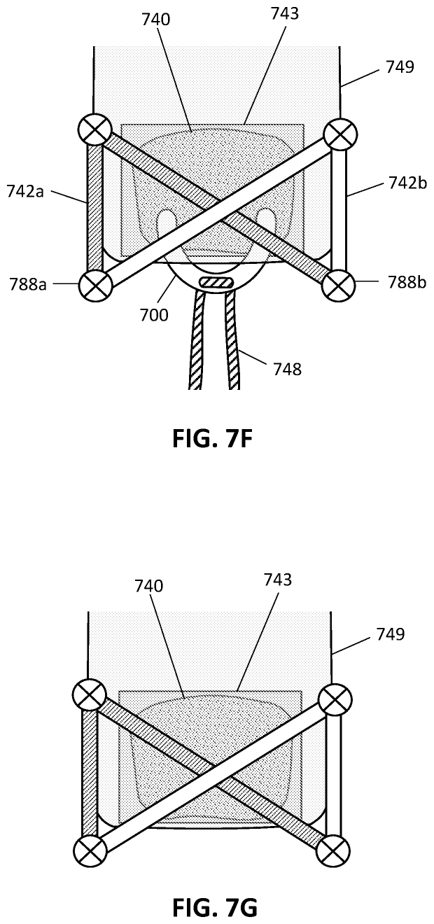

FIGS. 7A-7G illustrate an exemplary procedure for implanting a graft using a temporary removeable securement. FIG. 7A shows a decortication footprint 740 on a region of a bone (e.g., humerus) where an outer cortical layer of bone has been removed to reveal the vascularized, growth factor rich cancellous bone below. The decoration footprint 740 serves as the attachment site for the graft. In some cases, the decortication is performed deep enough to facilitate release of bone marrow components (e.g., blood, stem cells). In some cases, a marrow vents (e.g., microfractures) are optionally formed within the decorticated region to release bone marrow components. If microfractures are formed, the diameter of the holes and spacings between the holes may vary. For example, for rotator cuff repair, microfracture holes may have diameters of about 1 mm to 1.5 mm and spaced apart about every 2-3 mm.

FIG. 7B shows the graft 743 positioned over the decoration footprint/attachment site 740. The size and shape of the graft 743 may depend on the decortication footprint 740. For example, the decortication footprint 740 may be measured to determine its dimensions and the graft may be hydrated and cut to size accordingly. If needed, the graft may be folded so that is it not wider than a particular thickness (e.g., about 8 mm). In some cases, the graft 743 is loaded onto an atraumatic laparoscopic forceps for delivery to the attachment site 740. In some cases, a soft cannula is used to insert the graft 743 at the attachment site 740.

FIG. 7C shows a temporary removeable securement 700 implanted through the graft 743 and into the underlying decorticated bone 740, thereby holding the graft 743 in place at the attachment site 740. In this example, the removeable securement 700 includes two prongs 702a and 702b inserted in two locations within the graft 743 and decorticated bone 740, thereby providing two points of fixation to prevent rotation of the graft 743. In other cases, the retention retaining device may include one prong or more than two prongs (e.g., 3, 4, 5, 6, 7, 8, 9, 10 or more prongs). In some cases, more than one retention retaining device is inserted to temporarily fixate the graft 743. The removeable securement 700 should cover an adequate retention surface area to prevent pull-through of the prongs 702a and 702b. The prongs 702a and 702b should also be inserted to an adequate depth to prevent premature pullout of the 702a and 702b, yet not so deep as to prevent pull out during removal of the removeable securement 700.

The removeable securement 700 includes a tether 748 that loops through the removeable securement 700, as described herein. In some cases, proximal ends of the tether 748 extend extracorporeally. The tether 748 should have sufficient tensile strength to resist breakage when pulled on during removal of the removeable securement 700. The tether 748 may be made of any suitable material. In some cases, the tether is made of a synthetic non-absorbable material and have a diameter ranging from about 0.01 mm and 0.8 mm.

In some examples, the prongs 702a and 702b are inserted using a removeable securement insertion tool (e.g., tool 550) such that the holes through the bone are sufficiently deep to provide marrow venting. Thus, such holes can serve as marrow vents and retention holes for the prongs 702a and 702b of the removeable securement 700. In some examples, the diameters of the marrow vents range from about 0.8 and 1.5 mm. The depth of the marrow vents may vary depending, for example, how deep the decorticated surface 740 was made. In some instances, 1 mm deep holes are adequate for marrow venting. In some instances, the holes may be deeper, e.g., ranging from about 8 mm and 12 mm. In some cases, the marrow vents have depths that are greater than the length of the prongs 702a and 702b of the removeable securement 700.

FIG. 7D shows a tendon (e.g., rotator cuff tendon) pulled over the graft 743 and a portion of the retention insert 700. As shown, the removeable securement 700 can be positioned so that the U-shaped apex and tether 748 are laterally outside of an edge of the tendon 749 for easy removal of the removeable securement 700. In some case, the tendon is pulled using an arthroscopic positioning tool and/or any other techniques as known.

At FIG. 7E, medial anchors 741a and 741b are implanted into the bone at the medial edge of the decortication footprint 740. In this case, the medial anchor 741a includes a first tendon securing suture 742a attached thereto, and the medial anchor 741b includes a second tendon-securing suture 742b attached thereto.

FIG. 7F shows the tendon 749 secured by the tendon-securing sutures 742a and 742b. Any suturing technique may be used. In this example, the tendon-securing sutures 742a and 742b are crossed over the tendon 749 and secured with lateral anchors 788a and 788b, thereby securing the tendon 749 to the bone over the graft 743 and the underlying decortication footprint 740. A portion of the removeable securement 700 remains positioned between the graft 743 and the tendon 749. As shown, the removeable securement 700 can be positioned so that the U-shaped apex and sutures are laterally outside of the edge of the tendon 749 for easy removal of the removeable securement 700 after the tendon 749 is sufficiently secured.

Once the tendon 749 is sufficiently sutured in place, the tether 748 can be pulled to remove the temporary removeable securement 700 from under the tendon 749, as shown in FIG. 7G. The tether 748 may be pulled in a non-perpendicular direction with respect to a long axis of the prongs, or in a non-parallel direction with respect to the flange of the removeable securement 748 (e.g., down), as accommodated by the curvature of the bone and surrounding anatomical structure. This can cause the removeable securement 700 to rotate/pivot to an extent that the prongs are released from the retention holes. The tether 748 may be completely removed from the patient's body, thereby reducing concerns of foreign body response or tissue damage.

It should be noted that the procedure sequence presented in FIGS. 7A-7G is only exemplary and that the removeable securements described herein may be implemented in other manners. For example, in some cases, the removeable securement can be used to transiently secure the graft to the decorticated surface, then medial anchors may be inserted through the graft so that the removeable securement may be removed. Other examples and sequence of procedures may be implemented.

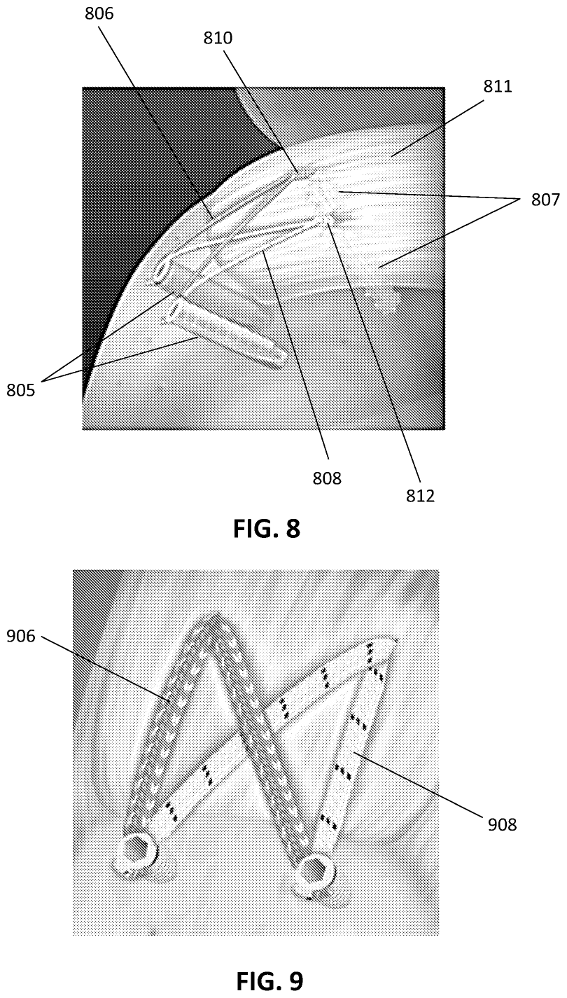

Any suture securement technique may be used to secure a graft under a tendon/ligament as described herein. For example, FIG. 8 illustrates an example where sutures 806 and 808 are attached to anchors 805 and 807 to secure the tendon 811 to the bone. In this case, the sutures 806 and 808 are configured in knots 810 and 812 over medial anchors 807. FIG. 9 illustrates another example where the sutures 906 and 908 are adapted for knotless securement. In some cases, the sutures 906 and 908 are made of a fiber tape.

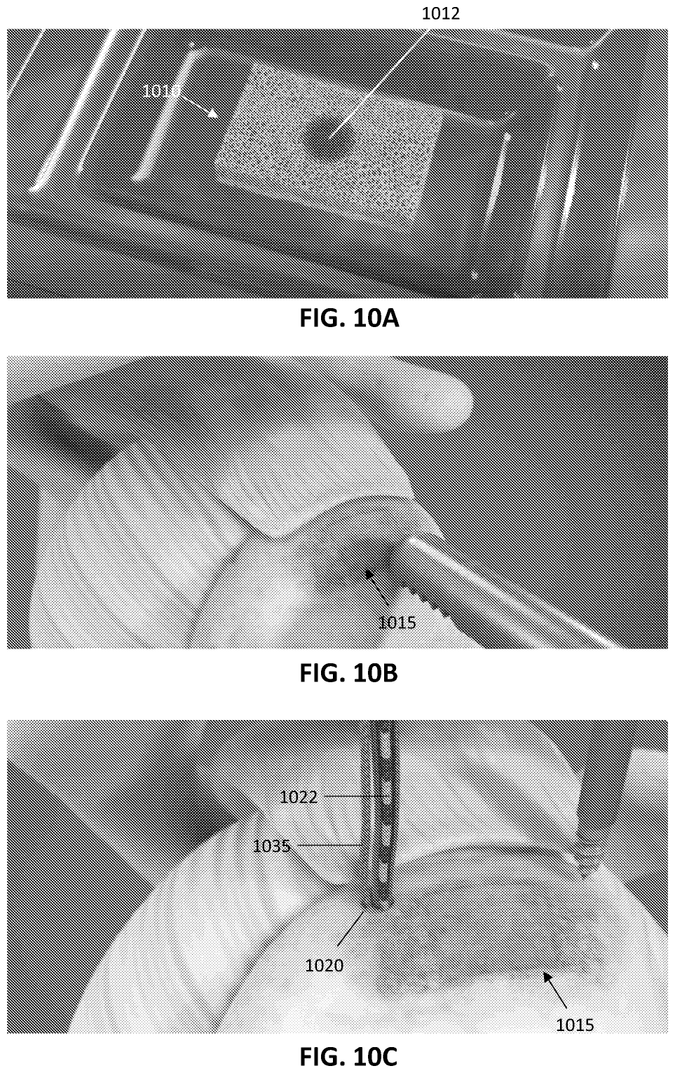

In any of the methods described herein both the graft and the region to be repaired may be prepared at the beginning of the procedure. For example, in any of these methods, the graft may be hydrated before implanting into the body. For example, FIG. 10A shows an example of a method of repairing an enthesis (e.g., in this example, a rotator cuff repair) as described herein. In general, the graft 1010 may be prepared ahead of the procedure, e.g., by hydrating it (e.g., in sterile water or isotonic buffer) for an appropriate time (e.g., >10 minutes, >15 minutes, etc.). In some examples the graft is hydrated in bone marrow aspirate and/or platelet-rich plasma. The graft may be marked, to indicate the superior and/or anterior surface. In FIG. 10A the graft includes a marking 1012 showing the superior (demineralized) face.

The bone surface may then be prepared, e.g., by decorticating the region onto which the graft is to be applied. FIG. 10B shows the bone surface being decorticated. As mentioned above, the bone and tendon may be accessed minimally invasively. This process may expose the marrow, so that marrow may flow into the graft when applied. Medial anchors may then be applied to the bone, on a region that is completely outside of the decorticated region 1015 of bone. This is shown in FIG. 10C. each anchor 1020 includes one or more sutures 1022.

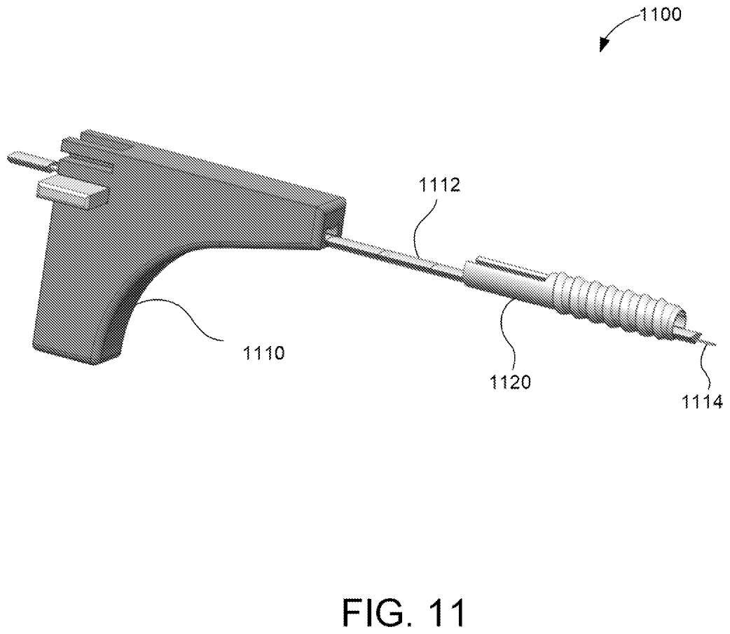

FIG. 11 is a simplified diagram of an implant delivery system 1100, in accordance with some examples. The implant delivery system 1100 may include an applicator handle 1110 and a cannula 1120. The applicator handle may include a shaft 1112 and a needle 1114. Although only one needle 1114 is shown here, in other examples, the implant delivery system 1100 may include any feasible number of needles. The cannula 1120 may be movably coupled to the shaft 1112.

The implant delivery system 1100 may be used to deliver implant material percutaneously to a patient. For example, the implant delivery system 1100 may be used to deliver and guide implant material into any region, including any feasible arthroscopic space. The implant material can be any feasible implant material. In some examples, the implant material can be a graft and/or a bone graft material that includes a layers of mineralized and demineralized material. The implant material may be porous, and in some cases different layers of the implant material may have different porosities. In some examples, the bone graft material may be configured to permit migration of bone marrow into the demineralized layer. In some other examples, the implant delivery system 1100 may guide and deliver bone graft material into the shoulder area to repair a rotator cuff.

The cannula 1120 may be separated from the applicator handle 1110 by, for example, sliding the cannula 1120 on the shaft 1112 distally (e.g., away from) the applicator handle 1110. When the cannula 1120 is separate from the applicator handle 1110, the cannula 1120 may be inserted into the patient. Following insertion, the implant material may be placed into (loaded) into the cannula 1120 and the shaft 1112 re-coupled to the cannula 1120.

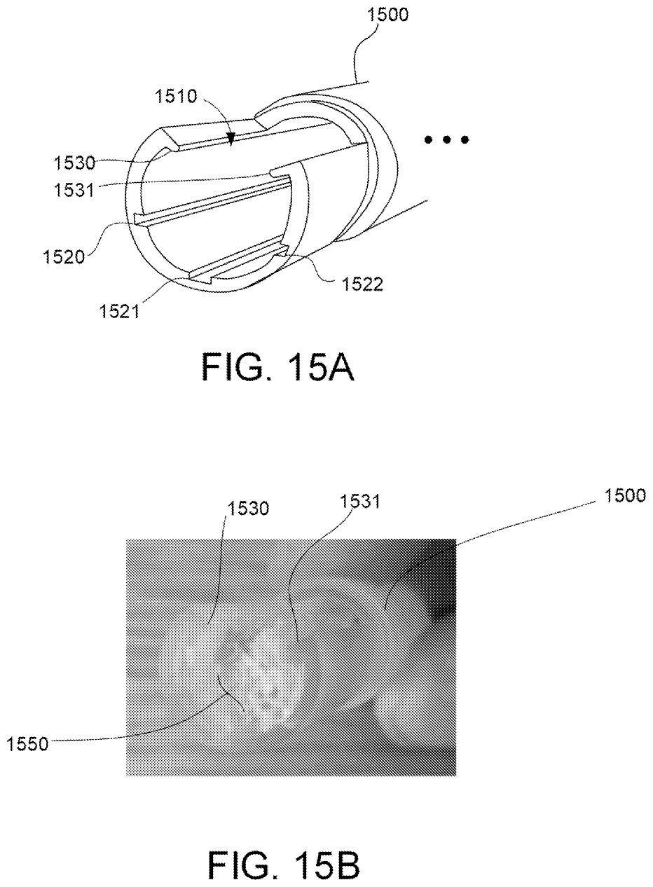

As the shaft 1112 is inserted into the cannula 1120, the needle 1114, which may protrude from the distal end of the shaft 1112, may penetrate and engage with the implant material. The needle 1114 may be enclosed and/or surrounded by the shaft 1112. Thus, the needle 1114 in combination with the shaft 1112 may be used to guide the implant material through the cannula 1120. In some examples, a clinician may guide the implant material into the arthroscopic space by manipulating the applicator handle 1110, shaft 1112, needle 1114, and/or the cannula 1120. In some cases, after delivery of the implant material to the implant region, the clinician may use the applicator handle 1110 to stabilize the implant material prior to and in some cases during fixation. Implantation and fixation of the implant material is described in more detail below in conjunction with FIGS. 15A-15C, 16, and 17.

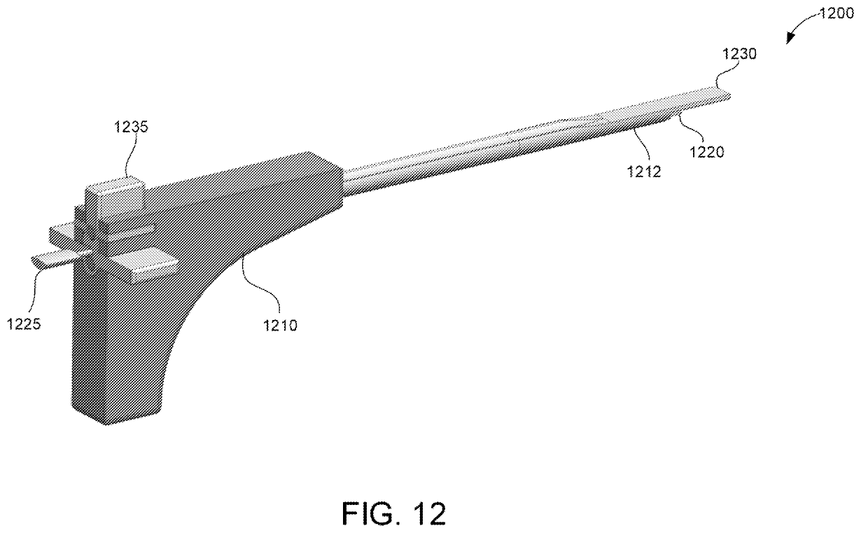

FIG. 12 is a simplified diagram of an applicator handle 1200, in accordance with some examples. The applicator handle 1200 may include a handgrip 1210 a shaft 1212, a needle 1220 and an extension arm 1230. The applicator handle 1200 may be an example of the applicator handle 1110 of FIG. 11.

As described with respect to FIG. 11, the applicator handle 1200 may be used with a cannula (not shown) to introduce and guide an implant to an arthroscopic space and, in some cases, stabilize a position of the implant while the implant is attached and/or fixated within the patient. The handgrip 1210 may be shaped to allow the clinician to grasp and control the applicator handle 1200 and/or the cannula particularly when the applicator handle 1200 is coupled with the cannula. The handgrip 1210 may be formed from any feasible material.

The shaft 1212 may be coupled to the handgrip 1210. The shaft 1212 may be rigid, partially rigid, or flexible. The shaft 1212 may be any feasible length and may be configured to slidably couple to the cannula. The shaft 1212 may extend distally away from the handgrip 1210. Thus, the shaft 1212 may be smooth and include features (rails, ridges, protrusions, or other feasible characteristics) to movably couple with the cannula. When the cannula is movably coupled to the shaft 1212, the clinician may control the applicator handle and cannula through the handgrip 1210.

The needle 1220 may traverse the length of the handgrip 1210 and the shaft 1212. As shown, a tip of the needle 1220 may extend from a distal (with respect to the handgrip 1210) end of the shaft 1212. A proximal end of the needle 1220 may include a needle controller 1225.

In some examples, the needle 220 may be flexible and may slide within the shaft 1212. For example, the needle 1220 may slide proximally and distally within the shaft 1212. In some cases, the clinician can control the amount of needle 1220 extending beyond the shaft 1212 by moving the needle controller 1225 either proximally or distally. The needle 1220 may be used to extend, at least partially, into implant material that may be positioned at a distal end of the shaft 1212. In some examples, the clinician can adjust the needle 1220 to be inserted approximately half way through the implant material. In some other examples, the needle 1220 may extend any feasible amount through the implant material. In this manner, the needle 1220 may be used to control positioning and placement of the implant material, particularly as the implant material is placed and fixated within a patient. In some examples, use of the needle 1220 may be optional.

The extension arm 1230 may be movably coupled to the handgrip 1210. In some cases, the extension arm 1230 may be inserted into the handgrip 1210 before or after the shaft 1212 is inserted into the cannula. Thus, the extension arm 1230 may be inserted with the shaft 1212 into the cannula. In some other cases, the extension arm 1230 may be inserted within the handgrip 1210 while being partially withdrawn. During implantation, the clinician may load implant material into the cannula push the handgrip 1210 and the shaft 1212 distally into the cannula. The motion of the shaft 1212 relative to the cannula can deploy and deliver the implant material from the cannula and into the patient. If the clinician has positioned a distal portion of the cannula near or within an arthroscopic space, then the clinician can deliver the implant material to the arthroscopic space. After the implant material has been deployed from the cannula, the clinician may push the extension arm controller 1235 distally causing a distal end of the extension arm 1230 to extend beyond the distal end of the shaft 1212. The distal end of the extension arm 1230 may be used to apply pressure to or otherwise position the implant material and thereby assist in fixating the implant material.

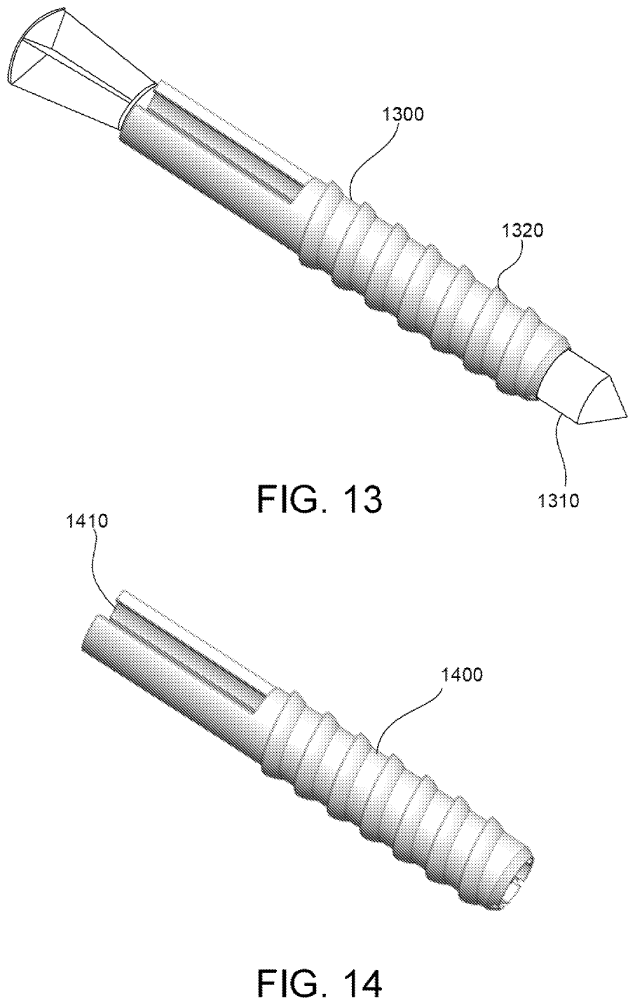

FIG. 13 is a simplified diagram of a cannula 1300, in accordance with some examples. The cannula 1300 may be an example of the cannula 1120 of FIG. 11. The cannula 1300 may include a removable obturator 1310 that may be inserted into the cannula 1300 and assist in the penetration of the cannula 1300 into the patent. For example, the obturator 1310 may be inserted into a proximal end of the cannula 1300 and protrude from a distal end of the cannula 1300. A distal end of the obturator 1310 may be sharp or blunt. As shown, the distal end of obturator 1310 may be pointed. The shape of the distal end of the obturator 1310 may aid in the penetration and percutaneous insertion of the cannula 1300.

In some examples, the cannula 1300 may include threads 1320 that may be affixed, attached, or in some cases molded onto the body of the cannula 1300. The threads 1320 may assist in the insertion of the cannula 1300 into the patient. For example, the clinician may twist the cannula 1300 enabling the threads 1320 to draw the cannula 1300 into the patient. The inner surface of the cannula 1300 may be smooth to facilitate smooth insertion and deployment of the implant material. The smooth inner surface may also enable the shaft of the applicator handle (not shown) to slide within the cannula 1300, The outer surface of the cannula 1300 may also be smooth. In some examples, the outer surface of the cannula 1300 may be textured to enable manipulation by the clinician, particularly during insertion of the cannula 1300. The texture finish may be achieved by diamond buffing, by abrasion with an abrasive such as sand paper or grit stone, and in some cases by bead blasting.

The cannula 1300 may be formed of any feasible material. In some cases, the cannula 1300 may be formed from impact resistant polymer that may be autoclavable. In some examples, the cannula 1300 and the obturator 1310 may be disposable or reusable.

FIG. 14 is another simplified diagram of a cannula 1400, in accordance with some examples. The cannula 1400 may be an example of the cannula 1120 of FIG. 11 and/or the cannula 1300 of FIG. 13. The cannula 1400 may include a slot 1410 located on or toward a proximal end of the cannula 1400. In some examples, the slot 1410 may be used to load the implant material into the cannula 1400. Loading of the cannula 1400 is described in more detail below in conjunction with FIGS. 15A-15C. In some examples, the cannula 1400 may include one or more features to facilitate placing (inserting) the implant material into the cannula 400. Some features are described below in conjunction with FIG. 15A.

FIG. 15A shows a proximal end view of a cannula 1500, in accordance with some examples. The cannula 1500 may include a slot 1510, shaft grooves 1520-1522, and retention cars 1530 and 1531. After the cannula 1500 is inserted into the patient (for example, as described with respect to FIGS. 13 and 14), the implant material, which may be roughly rectangular in shape, may be placed into the cannula 1500 through the slot 1510. For example, the implant material, which may be pliable, may be folded and inserted into the cannula 1500, folded end first. The folded end of the implant material may be placed into contact with the inner diameter of the cannula 1500, furthest from the slot 1510. In some cases, edges of the implant material may contact the retention cars 1530 and 5131. The retention cars 1530 and 1531 may “hold” the implant material within the cannula 1500, particularly when the implant material seeks to recover (e.g., spring back) into a flat, planar shape after being folded and placed into the cannula 1500.

After the implant material has been placed into the cannula 1500, the shaft portion of the applicator handle (not shown) may be inserted into the cannula. In some examples, the shaft grooves 1520-1522 may receive and/or engage with one or more features of the shaft. For example, the shaft may include rails, ridges, protrusions or the like that may be molded, affixed, or otherwise integrated into the shaft. Thus, the grooves 1520-1522 may enable the shaft to be movably coupled to the cannula 1500 and thereby guide the shaft through the cannula 1500. In some examples, the distal end of the shaft may engage with or contact the implant material. Thus, by moving the shaft distally relative to the cannula 1500, the implant material may be ejected or deployed from the cannula 1500. The distal end of the cannula 1500 may be positioned into the arthroscopic area by the clinician. Thus, the implant material may be moved from the cannula 1500 into a graft area that has be prepared to receive the implant material.

FIG. 15B shows another proximal end view of the cannula 1500, in accordance with some examples. The cannula 1500 may include the slot 1510 and the retention cars 1530 and 1531. As shown, the cannula 1500 may be loaded with implant material 1550. The retention cars 1530 and 1531 may help retain the implant material 1550 that has been placed within the inner diameter of the cannula 1500. For example, edges of the implant material 1550 may press against the retention cars 1530 and 1531 after the implant material 1550 has been inserted into the cannula 1500 through the slot 1510. FIG. 15C shows an example overhead view of the cannula 1500. The implant material 1550 is shown folded and placed through the slot 1510 within the cannula 1500. The implant material 1550 may have a rectangular shape.

The cannula 1500 may be used for delivery of the implant material 1550 into any feasible patient area including, without limitation, arthroscopic areas such as rotator cuff areas. If the cannula 1500 (for example, in conjunction with the implant delivery system 1100 of FIG. 11) is used to deliver the implant material through a lateral portal (e.g., an opening located on a coronal plane that includes the shoulder), then the implant material 1550 may be loaded into the cannula 1500 such that a long dimension of a rectangular shaped implant material 1550 may be placed in contact with the retaining cars 1530 and 1531. On the other hand, if the cannula 1500 is used to deliver the implant material 1550 through an anterior or posterior portal (e.g., an opening located on the chest or back of the patient), then the implant material 1550 may be loaded into the cannula 1500 such that a short dimension of the rectangularly shaped implant material 1550 may be placed in contact with the retaining cars 1530 and 1531.

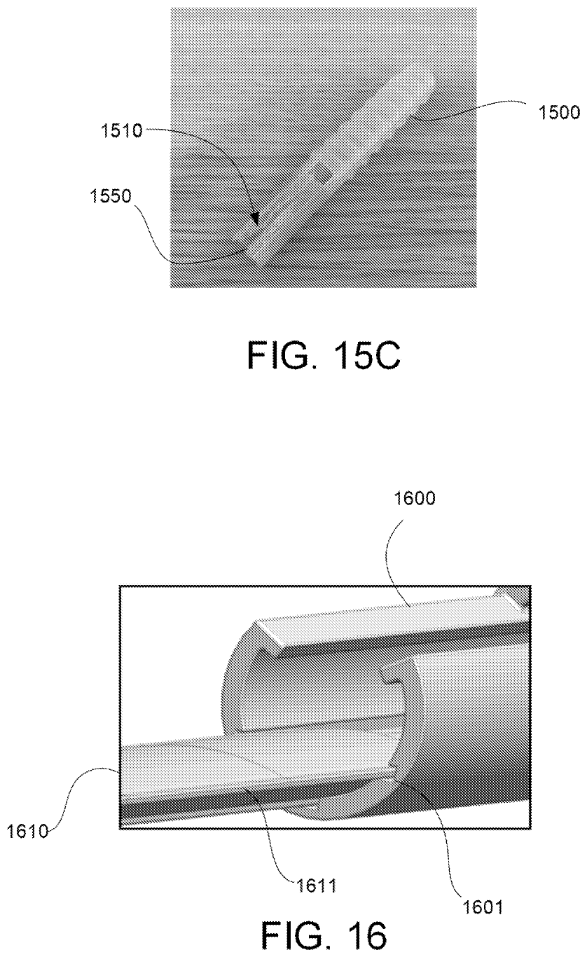

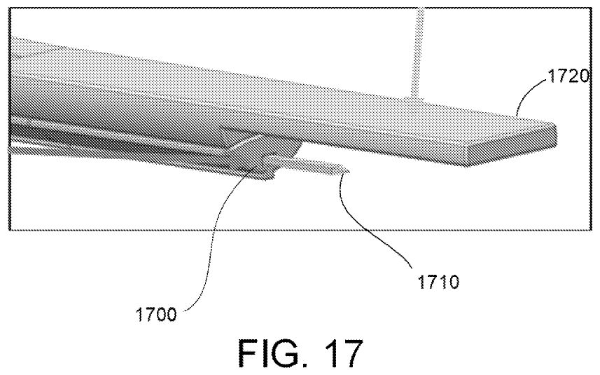

FIG. 16 shows a proximal end view of a cannula 1600 and a shaft 1610. As shown, the shaft 1610 may engage with the cannula 1600. For example, shaft 1610 may include one or more ridges 1611 that engage with related grooves 1601 included within the cannula 1600. The ridges 1611 and grooves 1601 may enable the cannula 1600 to be slidably (e.g., movably) coupled to the shaft 1610. Thus, the grooves 1601 and the ridges 1611 may cooperatively guide the motion of the shaft 1610 within the cannula 1600.

FIG. 17 shows a distal end view of the end of a shaft 1700, in accordance with some examples. The shaft 1700 may be an example of the shaft 1112 of FIG. 11, the shaft 1212 of FIG. 12, or the shaft 1610 of FIG. 16. The shaft 1700 may include a needle 1710 and an extension arm 1720. The needle 1710 may be an example of the needle 1114 of FIG. 11, or the needle 1220 of FIG. 12. The extension arm 1720 may be an example of the extension arm 1230 of FIG. 12.

As described above with respect to FIG. 12, the needle 1710 may be controlled by the clinician to extend at least partially into the implant material located at the distal end of the shaft 1700. For example, after the implant material is placed into the cannula (not shown), the shaft 1700 may be also inserted into the cannula. The clinician may control extension of the needle 1710 into the implant material by moving the needle controller (not shown) either distally or proximally within the shaft 1700.

In some cases, the distal face of the shaft 1700 may move the implant material through the cannula and into position within the patient. The clinician may cause the extension arm 1720 to move distally along the shaft 1700. As shown, the extension arm 1720 may extend beyond the distal face of the shaft 1700. In this manner, a distal portion of the extension arm 1720 may be placed in contact with the implant material. In some examples, the extension arm 1720 may be used to at least temporarily hold or apply pressure to the implant material while other implements or tools suture or otherwise affix the implant material to the patient. After the implant material is affixed to the patient, the extension arm 1720, needle 1710 and shaft may be withdrawn from the patient. The cannula may also be withdrawn. Width of a distal end of the extension arm may be comparable to, or associated with the distal face of the shaft 1700. An example width may be 9 millimeters (mm), however any feasible width is contemplated.

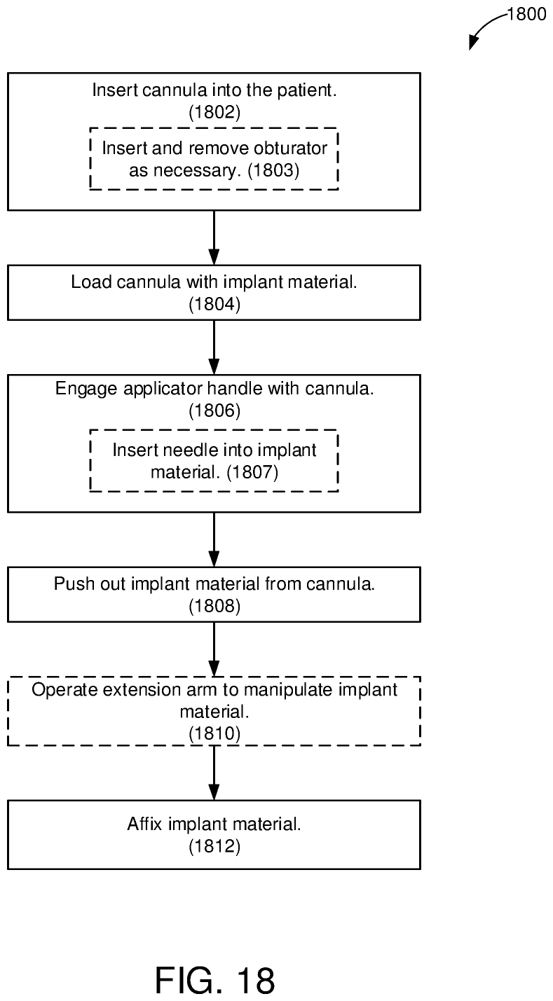

FIG. 18 is a flowchart showing an example method 1800 for delivering implant material into an implant region, in accordance with some examples. Some examples may perform the operations described herein with additional operations, fewer operations, operations in a different order, operations in parallel, and some operations differently. The method 1800 is described below with respect to the implant delivery system 1100 of FIG. 11, however, the method 1800 may be performed by any other suitable system or device.

The method 1800 begins in block 1802 as the cannula 1120 is inserted into the patient. For example, if implant material is bone graft material to be used in rotator cuff repair, then the clinician may insert the cannula 1120 through a lateral portal in the shoulder. In some other examples, the clinician may insert the cannula 1120 through the back or front (chest) of the patient.

In block 1803, an obturator (such as the obturator 1310 of FIG. 13) may optionally be used with the cannula 1120 to aid in the insertion of the cannula 1120. This optional step is illustrated with dashed lines in FIG. 18. For example, the clinician may insert obturator 1310 in the cannula 1120 prior to inserting the cannula 1120 into the patient. After insertion, the clinician can remove the obturator 1310 from the patient.

In block 1804, the clinician may load (place) implant material into the cannula. For example, the clinician can insert the implant material, which may be bone graft material, through a slot in the cannula 1120.

In block 1806, the clinician may engage the applicator handle 1110 with the cannula 1120. For example, a shaft 1112 of the applicator handle 1110 may be placed within the cannula 1120. In some cases, as the shaft 1112 enters the cannula 1120, a distal end of the shaft 1112 may contact the implant material. The implant material may move distally within the cannula 1120.

In block 1807, the clinician may optionally insert the needle 1114 into the implant material. For example, the clinician may manipulate a needle controller to insert the needle 1114 approximately half way through the implant material. If the implant material is 15 millimeters (mm) in length, then the clinician may insert the needle 1114 about 7.5 mm into the implant material. Any other needle insertion depths are possible. The needle 1114 may provide the clinician more control of the implant material through the applicator handle 1110 and the shaft 1112.

In block 1808, the clinician may push out the implant material from the cannula 1120. For example, the clinician may push the shaft 1112 distally with respect to the cannula 1120. This distal motion may cause a distal face of the shaft 1112 to push out the implant material from the cannula 1120 and into the patient.

In block 1810, the clinician may optionally operate the extension arm (such as the extension arm 1720 of FIG. 17) to manipulate the implant material. For example, by extending the extension arm 720, the clinician may place gentle pressure on the implant material. The pressure may stabilize the position of the implant material, thereby enabling an accurate fixation or attachment of the implant material to the patient.

In block 1812, the clinician may affix the implant material to the patient. In some examples, the clinician may suture the implant material to any feasible anatomical structure within the patient.

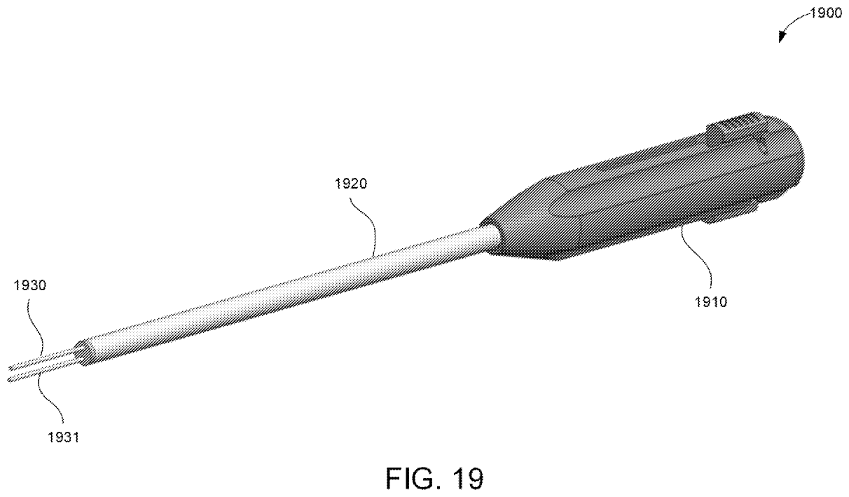

FIG. 19 is a simplified diagram of another implant delivery system 1900, in accordance with some examples. The implant delivery system 1900 may include a handle 1910, a shaft 1920, and engagement jaws 1930 and 1931. Although only two engagement jaws 1930 and 1931 are shown here, in other examples, the implant delivery system 1900 may include any feasible number of engagement jaws. As shown, the engagement jaws 1930 and 1931 may have a circular (round) cross section. In some examples, the cross section of the engagement jaws 1930 and 1931 may be flat, square, rectangular, ellipsoidal, trapezoidal, or any other feasible shape. The implant delivery system 1900 may be used to deliver and guide implant material, including bone graft material, into a region of the patient that has been prepared to receive the implant. Although not shown here, the implant delivery system 1900 may be used in conjunction with any feasible cannula and/or obturator. For example, the cannula and/or obturator may be used to guide, at least partially, the implant material to the implant region.

The handle 1910 may be coupled to the shaft 1920. The shaft 1920 may be rigid, partially rigid, or flexible. The shaft 1920 may extend distally from the handle 1910. The handle 1910 and the shaft 1920 may include openings and/or holes that enable the engagement jaws 1930 and 1930 to traverse the implant delivery system 1900. For example, the engagement jaw 1930 may be inserted into a first opening on the handle 1910 that is coupled to a first hole through the shaft 1920. Similarly, the engagement jaw 1931 may be inserted into a second opening on the handle 1910 that is coupled to a second hole through the shaft 1920. As the engagement jaws 1930 and 1931 are inserted into the handle 1910 and through the shaft 1920, respective ends of the engagement jaws 1930 and 1931 may protrude from (e.g., extend beyond) a distal face of the shaft 1920.

Implant material may be attached to (engage with) the ends of the engagement jaws 1930 and 1931. The implant delivery system 1900 may then be used to guide the implant material to the implant region. In some examples, the implant delivery system 1900 may be used to guide the implant material through a cannula to the implant region.

After the implant material has been placed in the implant region, the clinician may withdraw one of the engagement jaws 1930 or 1931 from the implant material. The implant material may then be affixed (in some cases sutured) to the patient. The single engagement jaw within the implant material may be used to stabilize and/or position the implant material. After the implant material has been affixed to the patient, the remaining engagement jaw 1930 or 1931 may be withdrawn from the implant material.

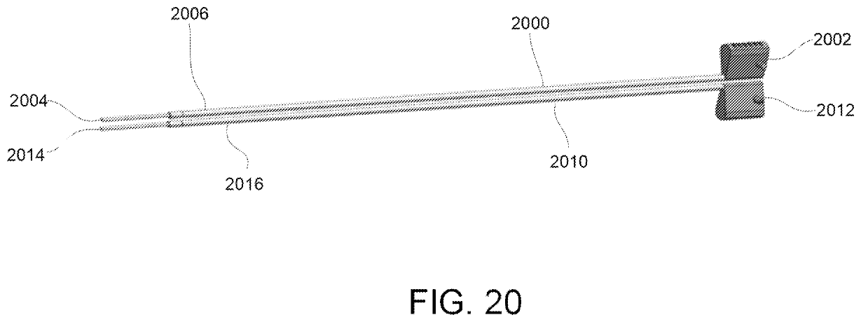

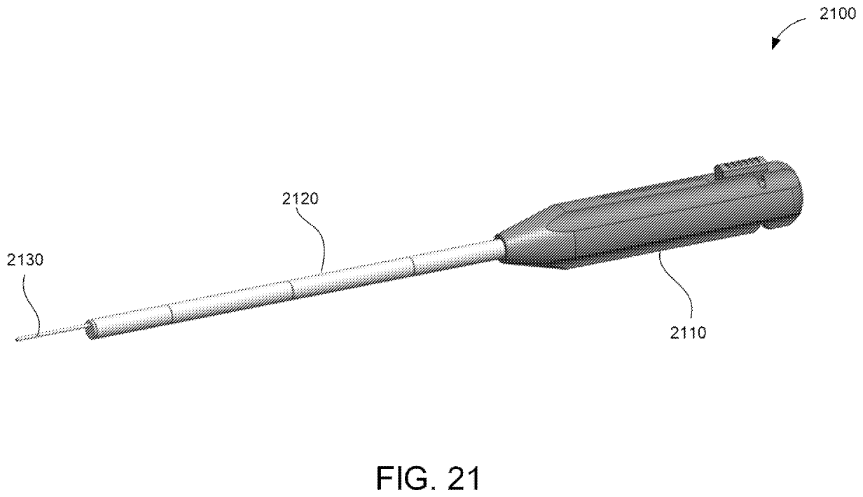

FIG. 20 shows engagement jaws 2000 and 2010, in accordance with some examples. The engagement jaws 2000 and 2010 may be examples of the engagement jaws 1930 and 1931 of FIG. 9. As shown, each of the engagement jaws 2000 and 2010 may be removable from the implant delivery system 1900 and/or the handle 1910. Further, each engagement jaw 2000 and 2010 may include grip, a rod, and an end. Thus, the engagement jaw 2000 may include a grip 2002, an end 2004, and a rod 2006 coupling the end 2004 to the grip 2002. Similarly, the engagement jaw 2010 may include a grip 2012, an end 2014, and a rod 2016 coupling the end 2014 to the grip 2012.

The grips 2002 and 2012 may be formed from any feasible material. In some cases, at least a portion of the grips 2002 and 2012 may include textured or grooved features to provide feasible surface for the clinician to manipulate the rods 2006 and 2016, respectively.

The rods 2006 and 2016 may be rigid or flexible. In some cases, the rods 2006 and 2016 may be formed from a flexible metal such as Nitinol. The ends 2004 and 2014 may be distally located on the rods 2006 and 2016 with respect to grips 2002 and 2012, respectively. In some examples, the cross section of the ends 2004 and 2014 may be circular, flat, square, rectangular, ellipsoidal, trapezoidal, or any other feasible shape. The engagement jaws 2000 and 2010 may be inserted into, and be movably coupled to the shaft 1920 of FIG. 19. In some examples, the rods 2006 and 2016 may slide within the shaft 1920 with a predetermined amount of friction. The ends 2004 and 2014 may include features that may enable the respective engagement jaws 2000 and 2010 to at least temporarily engage with the implant material.

FIG. 21 is a simplified diagram of another example implant delivery system 2100, in accordance with some examples. The implant delivery system 2100 may be similar to the implant delivery system 1900, but with the jaw 1931 removed. Thus, the implant delivery system 2100 may include a handle 2110, a shaft 2120, and an engagement jaw 2130. The handle 2110, the shaft 2120, and the engagement jaw 2130 may be examples of the handle 1910, the shaft 1920, and the engagement jaw 1930 of FIG. 19.

As described above, one of the jaws associated with the implant delivery system 2100 may be moved or removed. For example, a clinician can move a grip of an engagement jaw proximally away from a distal face of the shaft 2120. The remaining engagement jaw 2130 may enable the clinician to maintain control of the implant material while the implant or anatomy near the implant region are being manipulated to affix the implant material. Advantageously, removal of an engagement jaw from the shaft 2120 retracts the engagement jaw proximally from the implant material. That is, the implant delivery system 2100 may not impart any radial (with respect to a center of the shaft 2120) motion to the implant material when one or more of the engagement jaws are retracted. Lack of radial movement may allow the clinician to better control movement and placement of the implant material while being affixed.

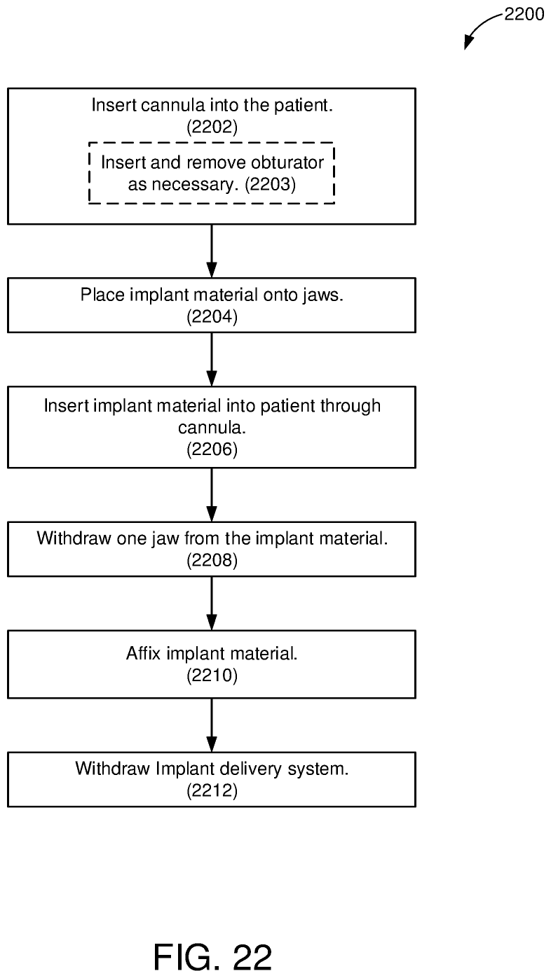

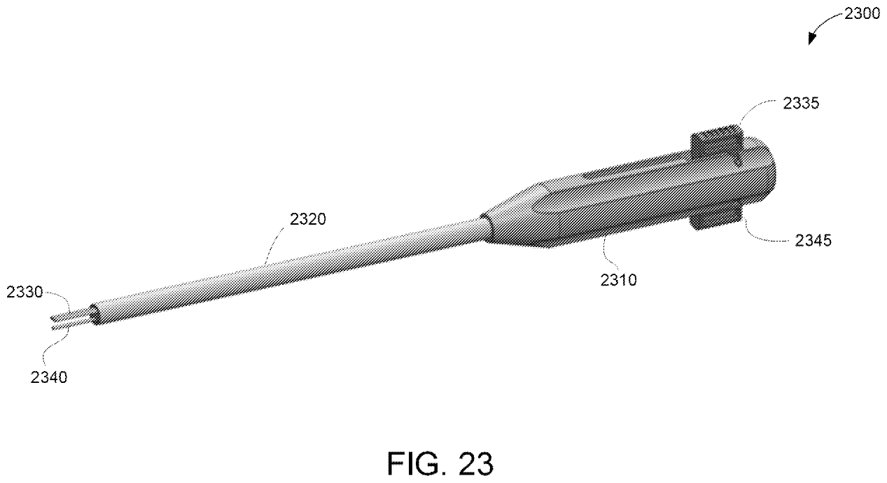

FIG. 22 is a flowchart showing an example method 2200 for delivering implant material into an implant region, in accordance with some examples. Some examples may perform the operations described herein with additional operations, fewer operations, operations in a different order, operations in parallel, and some operations differently. The method 2200 is described below with respect to the implant delivery system 1900 of FIG. 19, however, the method 2200 may be performed by any other suitable system or device.

The method 2200 begins in block 2202 as the cannula is inserted into the patient. For example, if implant material is bone graft material to be used in rotator cuff repair, then the clinician may insert the cannula through a lateral portal in the shoulder. In some other examples, the clinician may insert the cannula through the back or front (chest) of the patient.

In block 2203, an obturator may optionally be used with the cannula to aid in the insertion of the cannula. This optional step is illustrated with dashed lines in FIG. 22. For example, the clinician may insert an obturator in the cannula prior to inserting the cannula into the patient. After insertion, the clinician can remove the obturator from the patient.

In block 2204, the clinician places or attaches implant material onto the engagement jaws 1930 and 1931 of the implant delivery system 1900. In some examples, the engagement jaws may extend distally away from the handle 1910 and beyond the shaft 1920. Thus, the clinician may attach the implant material to the engagement jaws 1930 and 1931 that extend beyond the shaft 1920.

In block 2206, the clinician inserts the implant material into the patient through the cannula. In some examples, the clinician may place a distal end of the cannula into an implant region. Thus, as the clinician inserts the implant material through the cannula, the implant material may be near the region of the patient that has been prepared to receive the implant.

Next, in block 2208 the clinician withdraws one engagement jaw from the implant material. For example, the clinician may draw engagement jaw 1931 (by pulling or otherwise moving the engagement jaw 1931 proximally away from the implant material). In some cases, the clinician may press or actuate a grip, such as the grip 2012 shown in FIG. 20 to withdraw an engagement jaw from the implant material.

Next, in block 2210, the clinician affixes the implant material to the patient. In some cases, the clinician may suture or otherwise attach the implant material to any feasible structure or anatomy of the patient to affix the implant material. Advantageously, the implant delivery system 1900 may be used to control movement and/or placement of the implant material while the implant material is being affixed to the patient. In some cases, the clinician can control the implant material through the remaining engagement jaw in the implant delivery system 1900.

In block 2212, the clinician withdraws the implant delivery system 1900 from the patient. Since the implant material has been affixed in block 2210, the implant material is stable and the implant delivery system 1900 may be withdrawn.

FIG. 23 is a simplified diagram of another example implant delivery system 2300, in accordance with some examples. The implant delivery system 2300 may be similar to the implant delivery system 1900 of FIG. 19. The implant delivery system 2300 may include a cylindrical handle 2310, a shaft 2320 and engagement jaws 2330 and 2340.

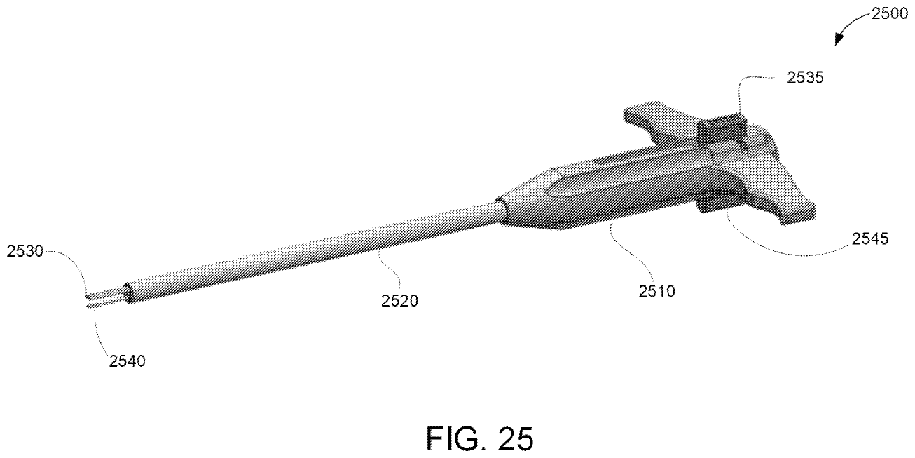

The engagement jaws 2330 and 2340 may be similar to the engagement jaws shown in FIG. 20. Thus, a grip 2335 may be coupled to a rod (not shown) and further coupled to the engagement jaw 2330. Similarly, a grip 2445 may be coupled to a rod (not shown) and further coupled to the engagement jaw 2340. As described below with respect to FIGS. 25A and 25B, the engagement jaw 2330 may include of three cylindrical features that may protrude from a distal face of the shaft 2320. Although shown here as three cylindrical features, in other examples, the engagement jaw 2330 may include any feasible plurality of cylindrical features. The engagement jaw 2340 may include a single cylinder feature protruding from the distal face of the shaft 2320.

A clinician may cause either or both engagement jaws 2330 and 2340 to withdraw into the shaft 2320 simply by pulling the grip 2335 and/or 2345 proximally (toward the clinician). In some cases, the clinician may remove a complete grip/rod/engagement jaw assembly from the handle 2310.

Similar to the implant delivery system 1900 of FIG. 19, implant material may be attached to (engage with) the ends of the engagement jaws 2330 and 2340. The implant delivery system 2300 may then be used to guide the implant material to the implant region. In some examples, the implant delivery system 2300 may be used to guide the implant material through a cannula to the implant region.

After the implant material has been placed in the implant region, the clinician may withdraw one of the engagement jaws 2330 or 2340 from the implant material. The implant material may then be affixed (in some cases sutured) to the patient. Similar to the applicator handle 1200 of FIG. 12, the clinician may use the extended engagement jaw 2330 to perform operations similar to the extension arm 1230. That is, the engagement jaw 2330 may be used to stabilize and/or position the implant material after the engagement jaw 2340 have been withdrawn from the implant region. After the implant material has been affixed to the patient, the engagement jaw 2330 may be withdrawn from the implant material and/or the implant region. In some examples, the implant delivery system 2300 may perform operations as described with respect to FIG. 22.

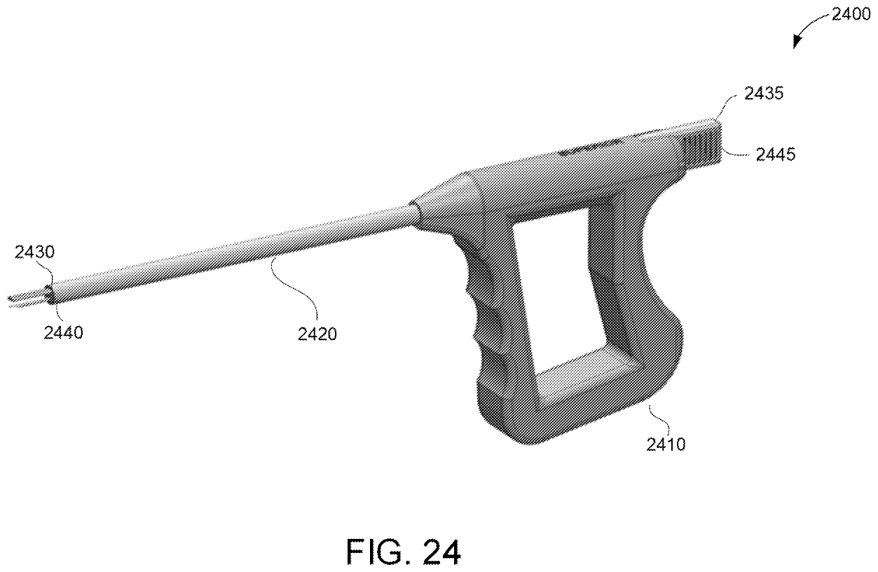

FIG. 24 is a simplified diagram of another example implant delivery system 2400. The implant delivery system 2400 may include a pistol grip handle 2410, a shaft 2420, and engagement jaws 2430 and 2440. The implant delivery system 2400 may be similar to the implant delivery system 2300. Thus, the shaft 2420, and engagement jaws 2430 and 2440 may be other examples of the shaft 2320 and engagement jaws 2330 and 2340 of FIG. 23. In some examples, the pistol grip handle 2410 may provide the clinician more comfort and control of the engagement jaws 2430 and 2440.

The handle 2410 may include grips 2435 and 2445. The grip 2435 may be coupled to a rod (not shown) and further coupled to the engagement jaw 2430. Similarly, the grip 2445 may be coupled to a rod (not shown) and further coupled to the engagement jaw 2440.

Implant material may be attached to (engage with) the ends of the engagement jaws 2430 and 2440. The implant delivery system 2400 may then be used to guide the implant material to the implant region. In some examples, the implant delivery system 2300 may be used to guide the implant material through a cannula to the implant region. The clinician may withdraw one of the engagement jaws from the implant region leaving the other engagement jaw to control the position of the implant material while being affixed to the patient. For example, the engagement jaw 2430 may be used to stabilize and/or position the implant material after the engagement jaw 2440 have been withdrawn from the implant region.

FIG. 25 is a simplified diagram of another example implant delivery system 2500. The implant delivery system 2500 may include a t-grip handle 2510, a shaft 2520, and engagement jaws 2530 and 2540. The implant delivery system 2500 may be similar to the implant delivery system 2300. Thus, the shaft 2520, and engagement jaws 2530 and 2540 may be other examples of the shaft 2320 and engagement jaws 2330 and 2340 of FIG. 23. In some examples, the t-grip handle 2510 may provide the clinician an alternative control surface that provides comfort and control of the engagement jaws 2530 and 2540.

The handle 2510 may include grips 2535 and 2545. The grip 2535 may be coupled to a rod (not shown) and further coupled to the engagement jaw 2530. Similarly, the grip 2545 may be coupled to a rod (not shown) and further coupled to the engagement jaw 2540.

Implant material may be attached to (engage with) the ends of the engagement jaws 2530 and 2540. The implant delivery system 2500 may then be used to guide the implant material to the implant region. In some examples, the implant delivery system 2500 may be used to guide the implant material through a cannula to the implant region. The clinician may withdraw one of the engagement jaws from the implant region leaving the other engagement jaw to control the position of the implant material while being affixed to the patient. For example, the engagement jaw 2530 may be used to stabilize and/or position the implant material after the engagement jaw 2540 have been withdrawn from the implant region.

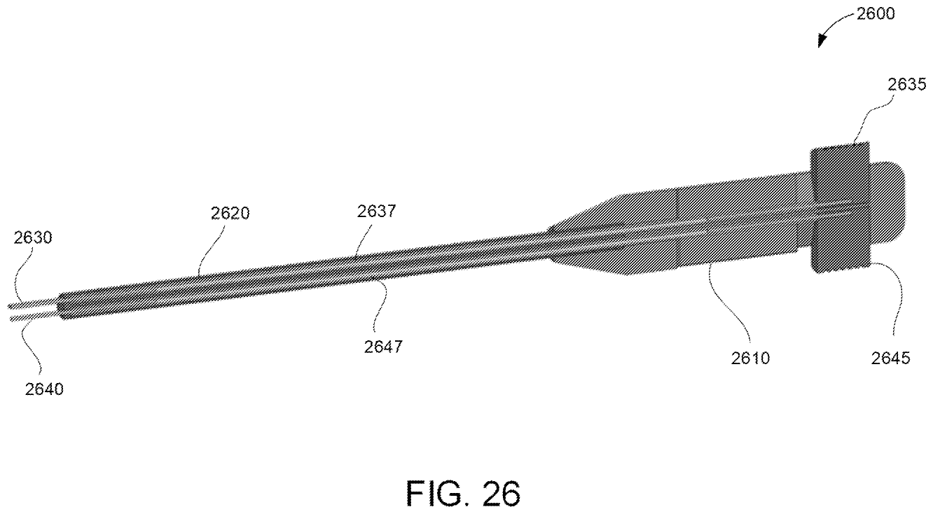

FIG. 26 is a cross section view of an implant delivery system 2600. The cross section may be associated with the implant delivery system 2300 of FIG. 23. However, there may be many similarities with the implant delivery system 1900 of FIG. 19, the implant delivery system 2400 of FIG. 24, the implant delivery system 2500 of FIG. 25, and other feasible implant delivery systems.

The implant delivery system 2600 may include a handle 2610, a shaft 2620 and engagement jaws 2630 and 2640. The engagement jaw 2630 may be coupled to a rod 2637 which is further coupled to a grip 2635. Similarly, the engagement jaw 2640 may be coupled to a rod 2647 which is further coupled to a grip 2645. In some examples, the rods 2637 and 2647 may be formed from Nitinol, or any other feasible material.

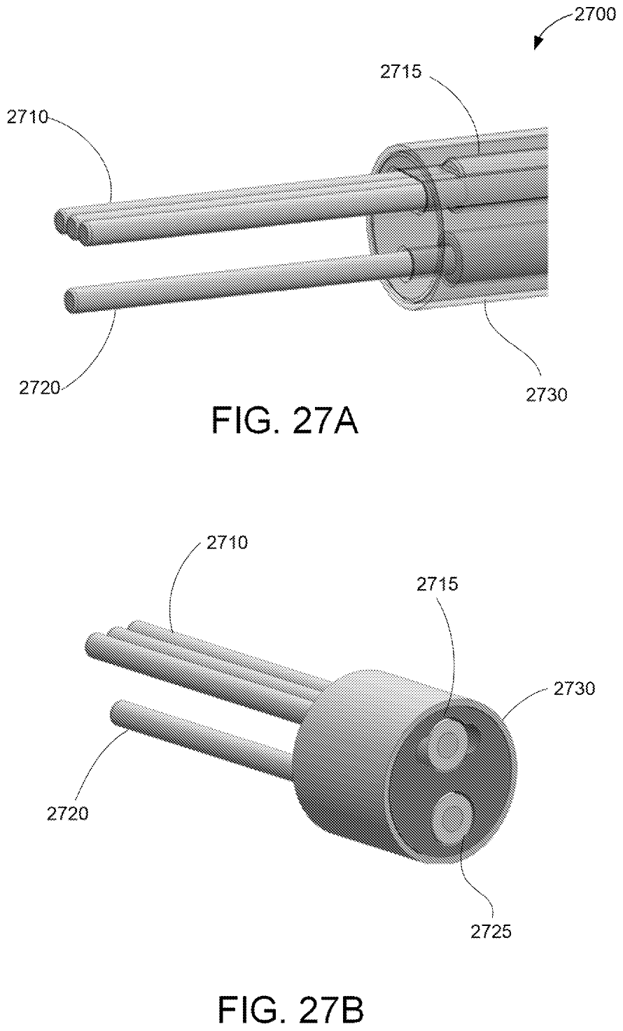

FIG. 27A shows a distal view of engagement jaws 2700. The engagement jaws 2700 may include a first engagement jaw 2710 and a second engagement jaw 2720. The first engagement jaw 2710 may be an example of the engagement jaw 2330 of FIG. 23, the engagement jaw 2430 of FIG. 24, the engagement jaw 2530 of FIG. 25, and/or the engagement jaw 2630 of FIG. 26.

As shown, the first engagement jaw 2710 may be formed of three discrete rods. Other examples may have more or fewer rods. The discrete rods may be arranged in a planar fashion providing a wider surface than a single rod. The wider surface may advantageously provide more control of the implant material while being affixed to the patient. The discrete rods may be coupled to a common rod to make actuation by a common grip feasible. For example, the first engagement jaw 2710 may be coupled to a single rod 2715 that traverses shaft 2730 and is coupled to a grip (not shown).

FIG. 27B shows a cross section view of the shaft 2730. Distal with respect to the cross section are the first engagement jaw 2710 and the second engagement jaw 2720. The individual rods that make up the first engagement jaw 2710 may be coupled to the rod 2715. In a similar manner, the second engagement jaw 2720 may be coupled to rod 2725. The rods 2715 and 2725 may be coupled to grips (not shown) that enable the clinician to individually move or articulate the respective engagement jaws.

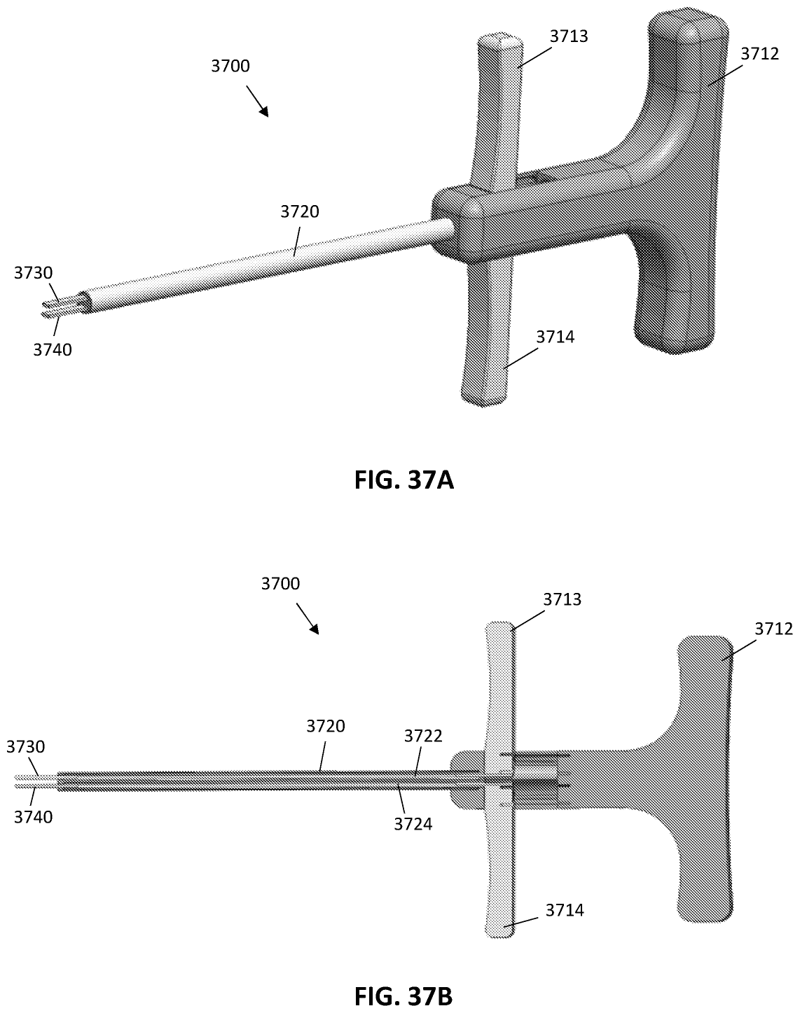

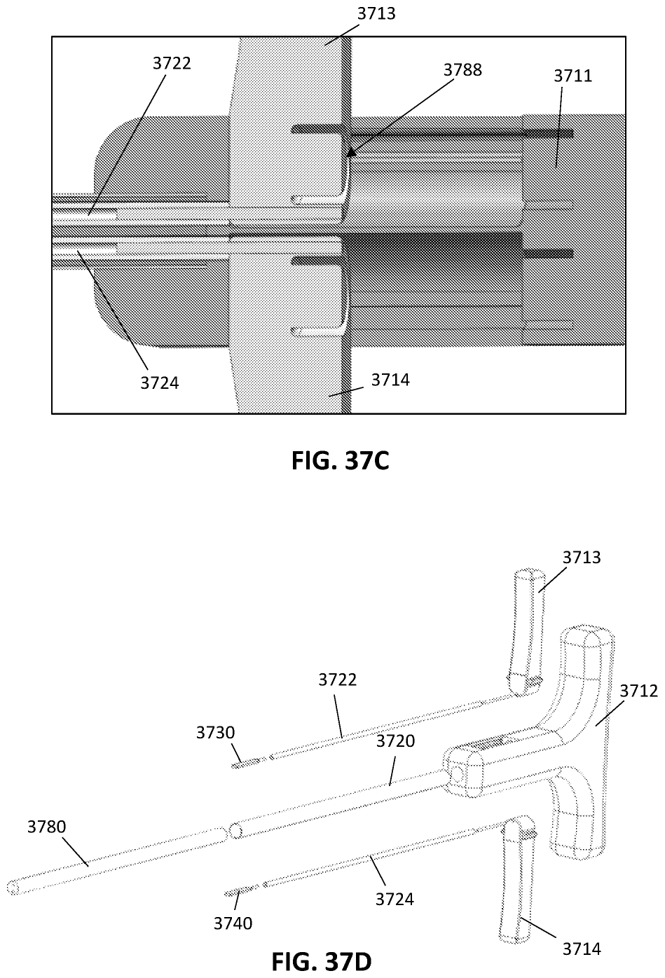

Another example of an implant delivery apparatus (e.g., configured as an implant delivery system) is shown in FIGS. 37A-37D. In this example, as in the examples shown and described above, the apparatus is configured to releasably hold an implant, e.g., a graft having a mineralized face that is adjacent to the mineralized face; the mineralized face may have a thickness that is less than a demineralized face of the graft. The apparatus may be configured to hold the implant between engagement jaws that may secure the graft between the jaws in a manner that prevents release of the jaws (e.g., holding the graft securely) without damaging the graft, by distributing the securing force between the jaws. The jaws may extend distally in-line from an elongate body, and the jaws may be configured to extend or retract to hold and release the implant, in the long axis of the elongate body. The elongate body may also extend from a proximal handle region that is configured to be handheld and operated with a single hand. For example, the apparatus may include a first finger-actuated control to retract (or in some examples extend) a flat jaw member at the distal end of the elongate shaft and a second finger-actuated control to retract (or in some examples extend) a second flat jaw member at the distal end of the elongate shaft in order to release (and in some examples to extend and capture) the implant. The proximal handle may also include a palm grip region. The proximal handle may also include a lock and a lock release. The lock may hold the first and second jaws in position to prevent accidental release of the implant when manipulating and positioning the implant (e.g., graft).

For example, FIG. 37A show a perspective view of an example of an implant delivery system 3700. In this example, the implant delivery system 3700 includes a palm grip handle portion 3712, a shaft 3720, and engagement jaws 3730 and 3740. The engagement jaws 3730 and 3740 are configured to be actuated by pulling on the finger controls 3713, 3714. Each finger control may be separately actuated, or in some configurations they may be coupled to withdraw (or in some examples extend) together as a unit. The first engagement jaw 3730 may be coupled to the first finger control 3713 and the second engagement jaw 3740 may be coupled to the second finger control 3714. Either or both finger controls may be coupled to a lock (not shown) that may prevent actuation of the first and/or second finger controls 3713, 3714. In some cases the long may be a finger or thumb release that may be on the palm grip handle 3712 or one or both finger controls. The lock may comprise a lever, toggle, switch or the like that may releasably engage with the finger control(s) and/or the elongate member 3722, 3724 connecting the finger controls to the engagement jaw(s). In some examples a single lock and lock release may be used; in some examples a pair of locks and/or lock releases may be used (e.g., one for each finger control). The palm grip handle 3712 may be configured to be comfortably gripped by the user and may be rounded or curved.

As shown in the sectional view of FIG. 37B, each finger control 3713, 3714 is coupled to a distal engagement jaw 3740, 3730 through an elongate member 3722, 3724. The elongate members may be wires, cables, rods, etc. The elongate members may be rigid or flexile.

The finger control(s) may be separately actuatable or jointly actuatable, as mentioned. In some example, actuation may move the finger controls distally or proximally relative to the palm grip (or palm support) 3712 portion, sliding the distal engagement jaws distally or proximally. In some examples a bias (e.g., spring, hydraulic, etc.) may be coupled to the finger control to bias the distal engagement jaws either proximally or distally. In some examples the apparatus may be configured to lock the control(s) for the distal engagement jaws in a first configuration (e.g., extended) until released, so that the distal engagement jaws may be securely held, preventing premature release of the implant (until the lock release is selected).

FIG. 37C shows an enlarged view of a portion of the proximal handle region showing the connection between the first finger control 3713 and the handle body 3711 and the second finger control 3714 and the handle body in this example. The first and second finger controls may slide distally or proximally relative to the handle body 3711. The first finger control 3713 is coupled to an elongate member 3722 configured as a push rod in this example, and the second finger control 3714 is coupled to the second elongate member 3724. In FIG. 37C, the handle body may include a cavity or internal mounting region for coupling to a bias (e.g., spiring). In some examples the apparatus may instead or additionally include a stop configured to operate as a hemostatic stop. In the example shown in FIGS. 37A-37D, the apparatus may be constructed of metal and polymeric martials that may be rigidly fixed (e.g., glued) to the articulating handles and/or plastic tubing.

The handle 3712 may include grips.

An exploded view of these elements is shown in FIG. 37D. The engagement jaws are coupled to the elongate member 3722 and 3724 and may be housed in an elongate inner member 3780 forming channels for each of the elongate members 3722, 3724. The inner member may fit within an outer elongate body 3720. For example, the outer elongate body may be formed of a stainless steel reinforcement sleeve.

An implant material may be secured or attached to (engage with) between the engagement jaws 3730 and 3740. As shown and as mentioned above, the engagement jaws may be configured as substantially flat, at least on one side. The engagement jaws may be elongate and configured to distribute the force securing the implant within the jaws. The engagement jaws may have the same configuration or may have different configurations. In some examples the engagement jaws may be configured to have a spatula-like body that is flat on at least the side configured to secure to the implant. In some examples the engagement jaw(s) may be rounded or ovoid in cross-section, e.g., forming rods. Thus, in any of these apparatuses, the jaws may be configured to increase contact footprint and arthroscopic security of the implant (e.g., graft).

In the apparatus shown in FIGS. 37A-37D, the device may be operated with a single hand. For example, the user may use a single hand to retract the jaw and release one jaw or both jaws to release the implant. In some examples it may be beneficial to release just a single jaw (e.g., retracting it proximally) so that the other jaw member may hold the implant in position within the body, such as against the bone region to which it is being secured until the second jaw can also be safely released without allowing migration (e.g., shifting of position) of the implant within the body.

In general, the implant delivery system 3700 may be used to guide and position an implant material to and within the implant region. In some examples, the implant delivery system may be used to guide the implant material through a cannula to the implant region. The clinician may withdraw one of the engagement jaws from the implant region leaving the other engagement jaw to control the position of the implant material while being affixed to the patient. For example, the engagement jaw 3730 may be used to stabilize and/or position the implant material after the engagement jaw 3740 have been withdrawn from the implant region.

As mentioned above, any of the apparatuses, including any of the implant delivery apparatuses (which may also be referred to herein as implant delivery systems or implant delivery devices) may be used with any appropriate access port or system, including a cannula system. These apparatuses may be adapted for use with off-the-shelf access ports or cannulas, including those having a valve to prevent release/removal of material from the body through the port or cannula.

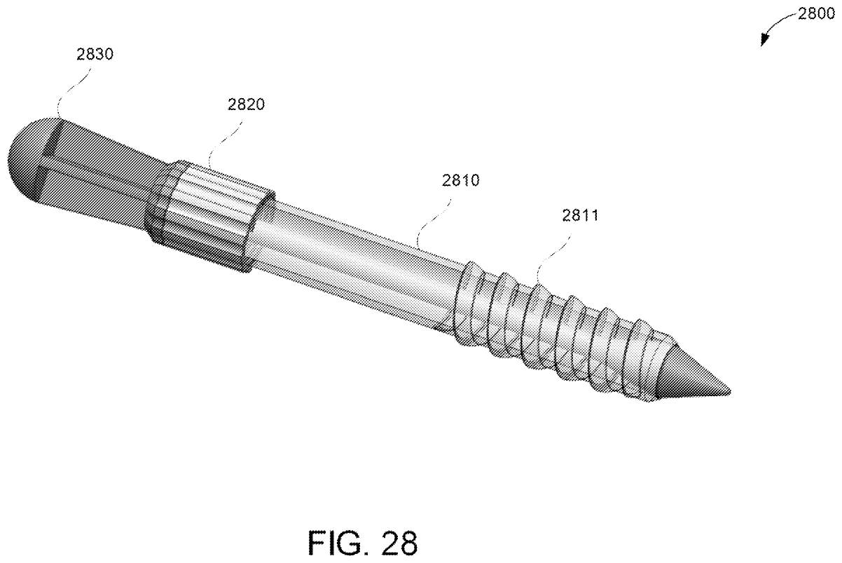

Returning now to FIG. 28, FIG. 28 shows a transiently liquid-tight cannula system 2800. The cannula system 2800 may an example of the cannula 1300 of FIG. 13, the cannula 1400 or FIG. 14, the cannula 1500 of FIGS. 15A-15C, the cannula 1600 of FIG. 16, or any other feasible cannula. The cannula system 2800 may be used in conjunction with the applicator handle 1200 of FIG. 12, the implant delivery system 1900 of FIG. 19, the implant delivery system 2300 of FIG. 23, the implant delivery system 2400 of FIG. 24, the implant delivery system 2500 of FIG. 25, or any other feasible device or system. The cannula system 2800 may provide a liquid-tight portal that may be used to deliver implant material to an implant region. For example, the cannula system 2800 may be inserted into a patient prior to receiving an implant. While in place, the cannula system 2800 may prevent or reduce fluid loss from any associated incision.

The cannula system 2800 may include a cannula 2810, a removable dam assembly 2820, and a removable obturator 2830. The cannula 2810 may generally be cylindrical in shape and have a proximal end (e.g., an end generally near the user, operator, clinician, etc.) and a distal end opposite the proximal end. The removable dam assembly 2820 may be coupled to the proximal end of the cannula 2810. The distal end of the cannula 2810 may include threads 2811 disposed on an outer surface to assist the user in inserting the cannula system 2800 through one or more layers of tissue. For example, the user may twist the removable obturator 2830 causing the cannula 2810 to twist and enabling the threads 2811 to draw the cannula 2810 into the tissue.

The removable obturator 2830 may be inserted through the removable dam assembly 2820 and through the cannula 2810. A distal end of the removable obturator 2830 may have a conical shape. Thus, when the removable obturator 2830 is inserted into the cannula system 2800, the distal end of the removable obturator 2830 may help insert the cannula system 2800 into the patient.

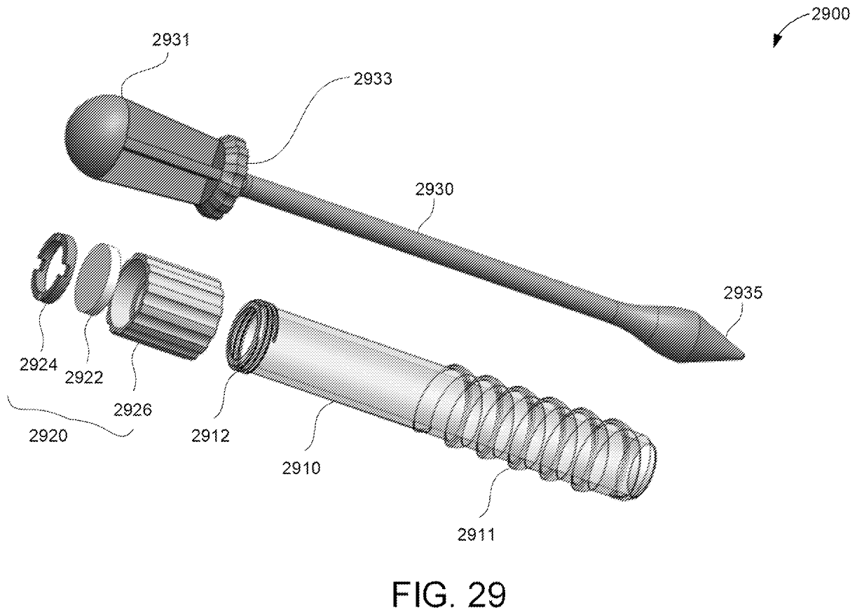

The removable dam assembly 2820 may prevent or reduce fluid loss when the cannula 2810 is inserted into the patient and will be described in more detail in conjunction with FIG. 29.

FIG. 29 shows an exploded view a cannula system 2900. The cannula system 2900 may be an example of the cannula system 2800 of FIG. 28 and may include a cannula 2910, a removable dam assembly 2920, and a removable obturator 2930. The removable obturator 2930 may include a handle 2931 (located proximally) and a conical point 2935 (located distally).

The cannula 2910 may include threads 2911 on a distal end to assist in the insertion and placement of the cannula 2910 with respect to the patient. In addition, the cannula 2910 may include threads 2912 disposed on a proximal end. The threads 2912 may be used to couple or attach the removable dam assembly 2920 to the cannula 2910.

The removable dam assembly 2920 may include a conforming gasket 2922, an engagement surface 2924, and a housing 2926. The housing 2926 may include threads (not shown) to engage with the threads 2912 on the cannula 2910. In this manner, the removable dam assembly 2920 may be attached to the cannula 2910. In addition, the housing 2926 may receive the conforming gasket 2922 and the engagement surface 2924. The conforming gasket 2922 may act as a barrier to prevent or reduce fluid loss through the cannula system 2900 when inserted in a patient. For example, when the removable dam assembly 2920 is affixed to the cannula 2910, the conforming gasket 2922 may block the flow of fluids through the cannula 2910. In some examples, the removable obturator 2930 may pierce the conforming gasket 2922. The conforming gasket 2922 may continue to provide a liquid-tight seal, even with the removable obturator 2930 in place. The conforming gasket 2922 may be formed from rubber, vinyl, any polymer, or any other feasible material.

The engagement surface 2924 may hold the conforming gasket 2922 within the housing 2926. In addition, the engagement surface 2924 may provide a surface for the removable obturator 2930 to engage with. In some examples, the removable obturator 2930 may have an engagement surface 2933 distal to the handle 2931. When the removable obturator 2930 is inserted into the cannula system 2900, the engagement surface 2933 may engage with the engagement surface 2924. In this manner, the removable obturator 2930 may be detachably coupled to the removable dam assembly 2920 and the cannula 2910. This coupling (e.g., engagement) allows twisting motion imparted to the handle 2931 to be coupled to the cannula 2910 through the removable dam assembly 2920.

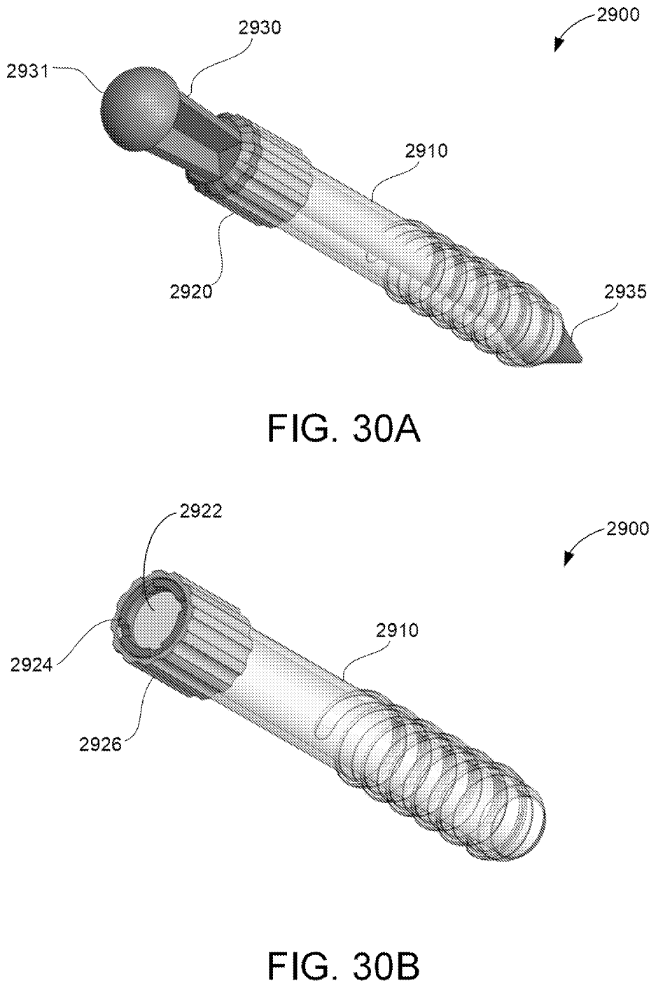

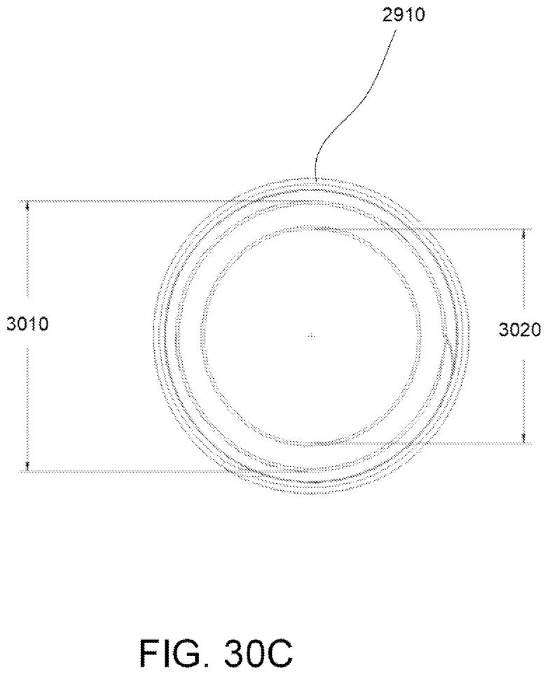

FIGS. 30A-30C show additional images of the cannula system 2900 of FIG. 29. FIG. 30A shows the cannula system 2900 with the removable obturator 2930 disposed through the cannula 2910 and the removable dam assembly 2920. FIG. 30B shows the cannula system 2900 with the removable obturator 2930 removed. The removeable dam assembly 2920 is shown affixed to the cannula 2910. The conforming gasket 2922 is shown with the engagement surface 2924. FIG. 30C shows example dimensions that may be associated with the cannula 2910. An outer diameter 3010 of the cannula 2910 may be 12 millimeters (excluding any threaded regions) and an inner diameter 3020 of the cannula 2910 may be 9.5 millimeters (excluding any threaded regions). The dimensions described herein are merely exemplary and are not meant to limit any implementations. In other implementations, the inner and outer diameters may be any feasible dimension.

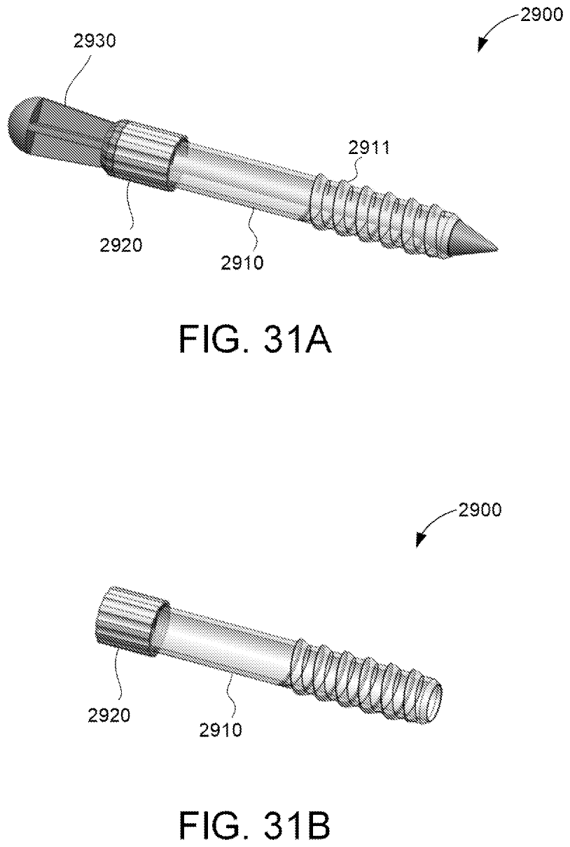

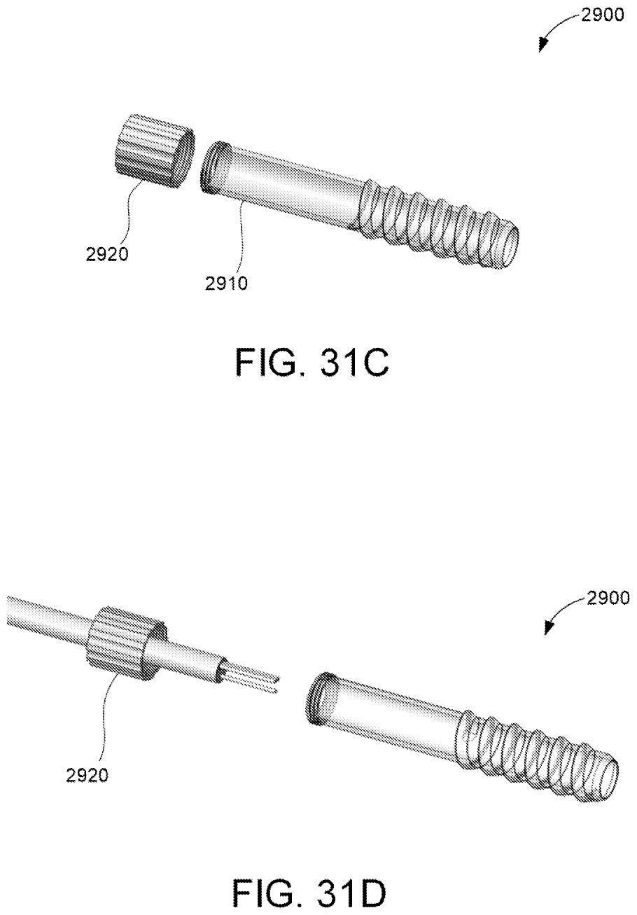

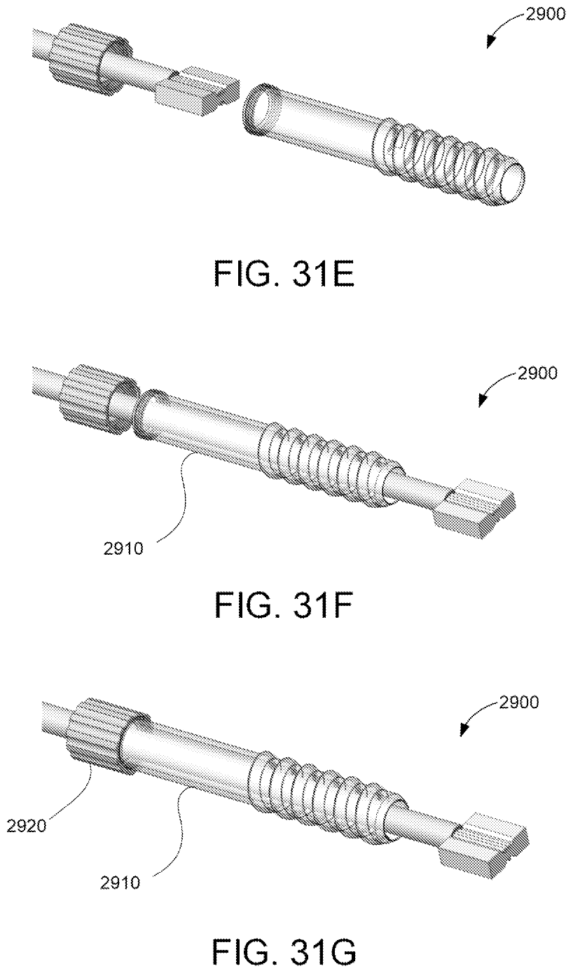

FIGS. 31A-31F illustrate different configurations of the cannula system 2900 of FIG. 29 while being used to deliver implant material to an implant region. FIG. 31A shows the cannula system 2900 with the removable dam assembly 2920 attached to the cannula 2910 and the removable obturator 2930 disposed through the removable dam assembly 2920 and the cannula 2910. While in this initial configuration, the user may insert the cannula system 2900 into a surgical portal (e.g., an incision) on the patient. In some examples, the user may twist the removable obturator 2930 which, in turn, may twist the cannula 2910. This twisting action may enable the threads 2911 on the cannula 2910 to draw the cannula system 2900 into tissues of the patient.

FIG. 31B shows the cannula system 2900 after insertion into the tissues of the patient. At this point, the functions of the removable obturator 2930 (not shown) are no longer needed and the removable obturator 2930 may be removed. For example, the removeable obturator 2930 may be withdrawn in a proximal direction from the cannula system 2900. Since the removable dam assembly 2920 remains attached to the cannula 2910, a liquid-tight seal is provided to cannula system 2900.

FIG. 31C shows the cannula system 2900 as preparations are being made to insert implant material into patient. First, the removable dam assembly 2920 is detached from the cannula 2910. In some examples, the removable dam assembly 2920 may be unthreaded from the cannula 2910.

FIG. 31D shows the removable dam assembly 2920 having received a shaft of an implant delivery system. Example implant delivery systems may include the implant delivery system 1900 of FIG. 19, the implant delivery system 2300 of FIG. 23, the implant delivery system 2400 of FIG. 24, the implant delivery system 2500 of FIG. 25, or any other feasible device or system. As shown, engagement jaws may protrude from the shaft. FIG. 31E shows the cannula system 2900 as implant material attached to the engagement jaws of the implant delivery system. FIG. 31F shows the cannula system 2900 as the shaft of the implant delivery system is inserted through the cannula 2910. As shown, the implant material may extend through the cannula 2910. Next, in FIG. 31G the cannula system 2900 is shown as the removable dam assembly 2920 is re-attached to the cannula 2910. The reattachment may reestablish a liquid-tight seal with the cannula 2910. In this state, the user may affix or attach the implant material to the patient while the removable dam assembly 2920 helps retain liquids within the patient.

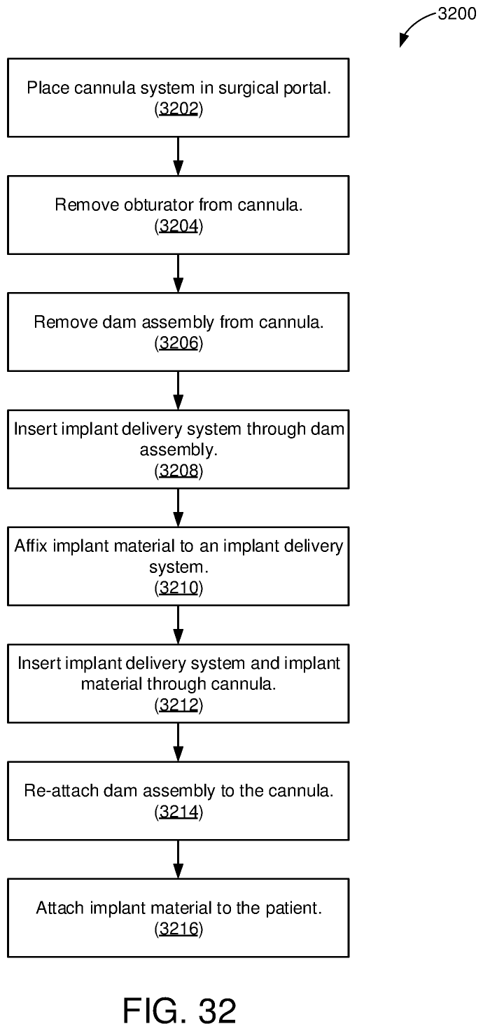

FIG. 32 is a flowchart showing an example method 3200 for delivering implant material into an implant region, in accordance with some examples. The method 3200 is described below with respect to the cannula system 2900 of FIG. 29, however, the method 3200 may be performed the cannula system 2800 of FIG. 28 or by any other suitable system or device.

The method 3200 begins in block 3202 as the cannula system 2900 is placed within a surgical portal. For example, the user may create a surgical portal by making an incision on the patient. After the incision is made, a distal end of the cannula system 2900 may be placed on, near, or within the surgical portal. The user may then twist the removable obturator 2930 causing the cannula 2910 to rotate and draw the cannula system 2900 into the surgical portal.

Next, in block 3204 the removable obturator 2930 is removed from the cannula 2910. Removal of the removable obturator 2930 leaves the removable dam assembly 2920 affixed to the cannula 2910 thereby providing a liquid-tight seal for the cannula system 2900.

Next, in block 3206 the removable dam assembly 2920 is removed from the cannula 2910. In block 3208 an implant delivery system is inserted through the removable dam assembly 2920. For example, engagement jaws configured to hold implant material may be inserted through the removable dam assembly 2920. In some cases, the engagement jaws may be inserted through the conforming gasket 2922. In block 3210 implant material may be attached or affixed to the implant delivery system. In some examples, the implant material may be attached to engagement jaws protruding form an end of a shaft of the implant delivery system.

Next, in block 3212 the implant delivery system and the implant material may be inserted through the cannula 2910. In block 3214, the removable dam assembly 2920 is re-attached to the cannula 2910. Reattachment of the removable dam assembly 2920 reestablishes a liquid-tight seal to the cannula 2910. Next, in block 3216 the user can attach the implant material to the patient.

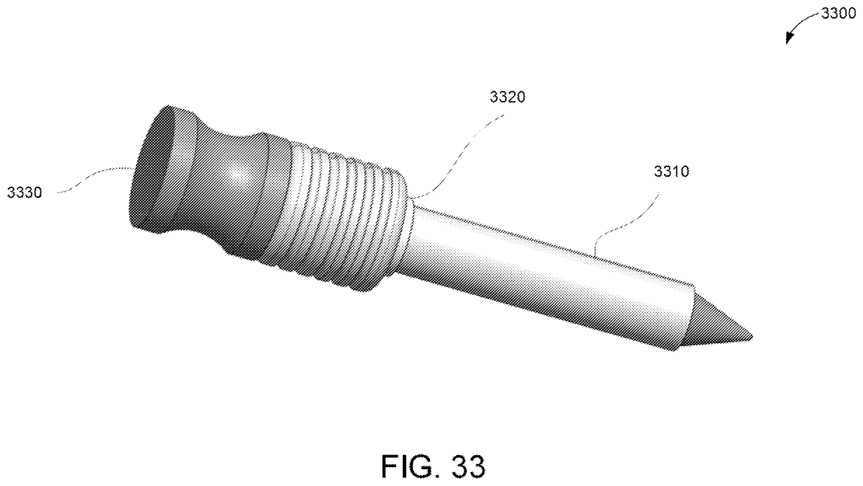

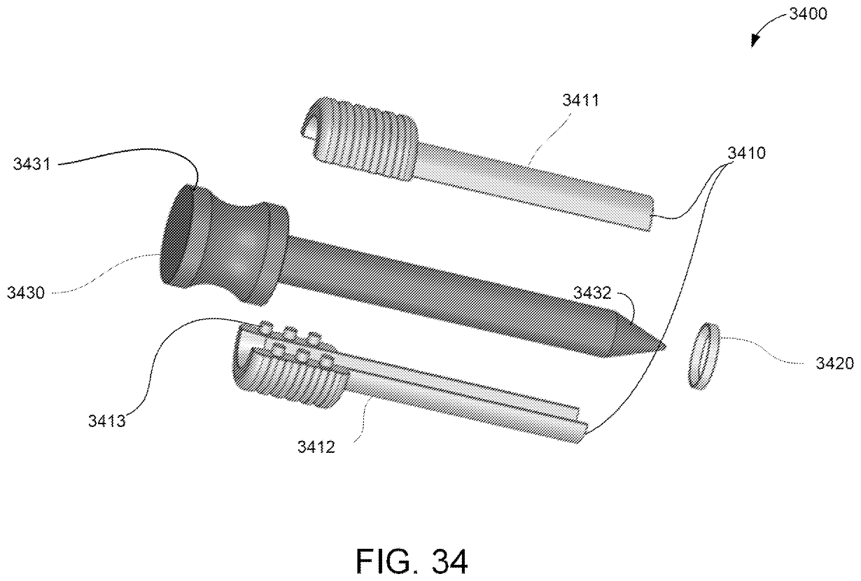

FIG. 33 shows a splitable cannula system 3300. The splitable cannula system 3300 may be used in conjunction with an implant delivery system, including any implant delivery system described herein. The splitable cannula system 3300 may provide a transient liquid-tight seal for a portal that may be used to insert and attach implant material to a patient.

The splitable cannula system 3300 may include a splitable cannula 3310, a removable fixation 3320, and a removable obturator 3230. The splitable cannula 3310 may be used to provide a cylindrical opening or tube for use with an implant delivery system (sometimes referred to as an applicator). In addition, the splitable cannula 3310 may separate into a number of pieces to allow removal of the splitable cannula 3310 while in used with the implant delivery system. The splitable cannula 3310 may include a distal end 3311 and a proximal end 3312. The distal end 3311 may inserted into a patient, in some cases through an implantable access portable. The proximal end 3312 may include knurling or other features that enable the user to grip the splitable cannula 3310.

The removable fixation 3320 may be used to hold the splitable cannula 3310 together, at least temporarily. For example, the removable fixation 3320 may hold the splitable cannula 3310 together while being inserted into an implantable access portal. In some examples, the removable fixation 3320 may be a weak adhesive, through other implementations are contemplated.

The removable obturator 3330 may fit snugly within the splitable cannula 3310 and provide a liquid-tight seal. The removable obturator 3330 may assist in placement of the splitable cannula system 3300. For example, a distal end of the removable obturator 3330 may include a conical or pointed end to help guide the splitable cannula system 3300 into position or into an implantable access portal.

FIG. 34 shows an exploded view a splitable cannula system 3400. The splitable cannula system 3400 may be an example of the splitable cannula system 3300 of FIG. 33 and may include a first cannula section 3411, a second cannula section 3412, a removable fixation 3420, and a removable obturator 3430. The first cannula section 3411 and the second cannula section 3412 together may form a splitable cannula 3410. Although only two sections are shown here, in other examples, the splitable cannula 3410 may include any number of sections. In some examples, the splitable cannula 3410 may include locating pins 3413 and corresponding locating sockets (not shown) to align the first cannula section 3411 to the second cannula section 3412. The removable obturator 3430 may include a handle 3431 (located proximally) and a conical point 3432 (located distally).

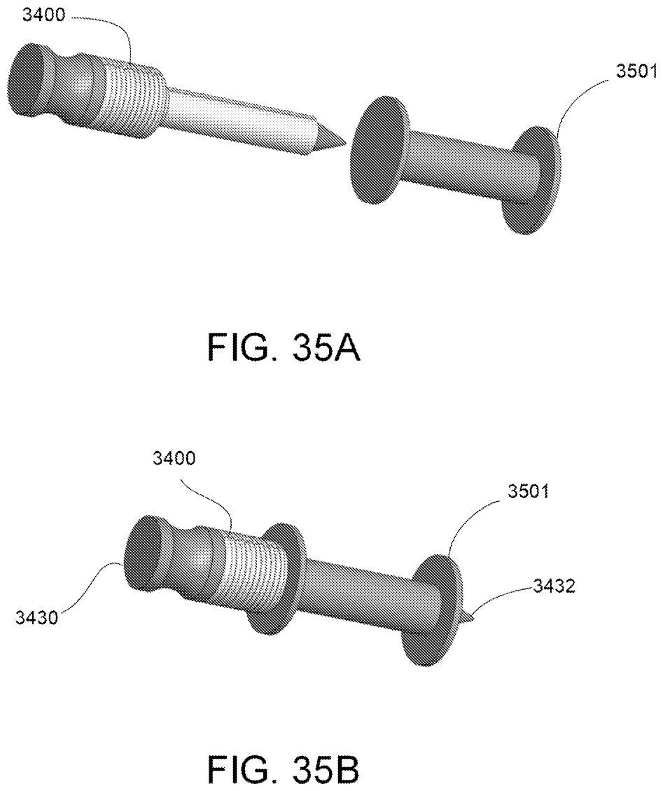

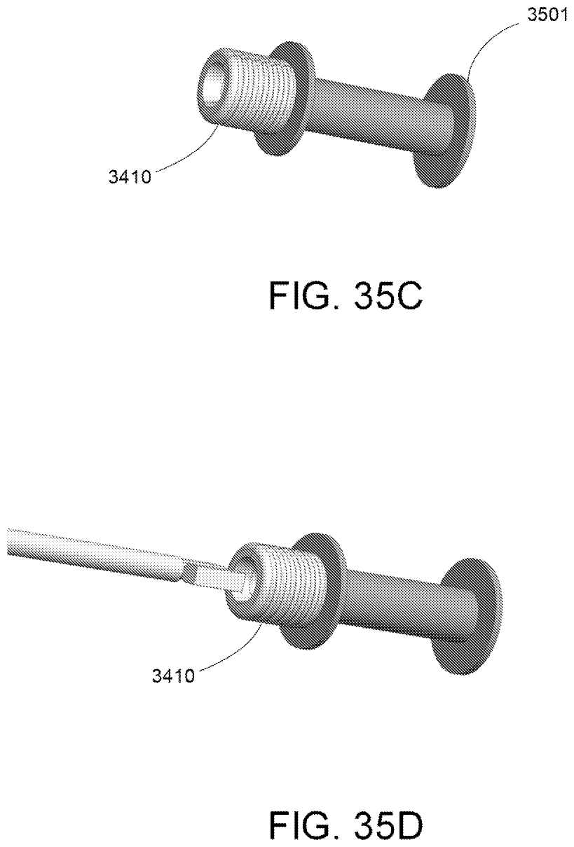

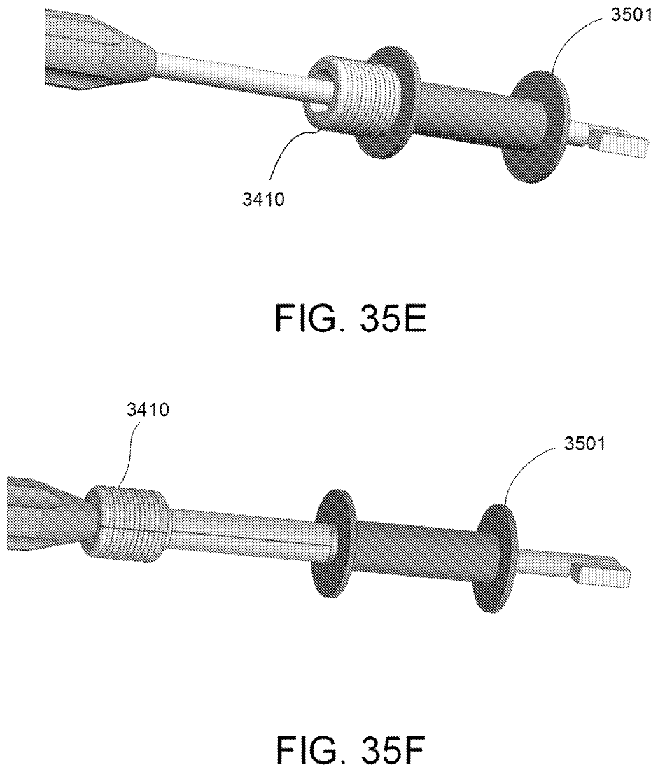

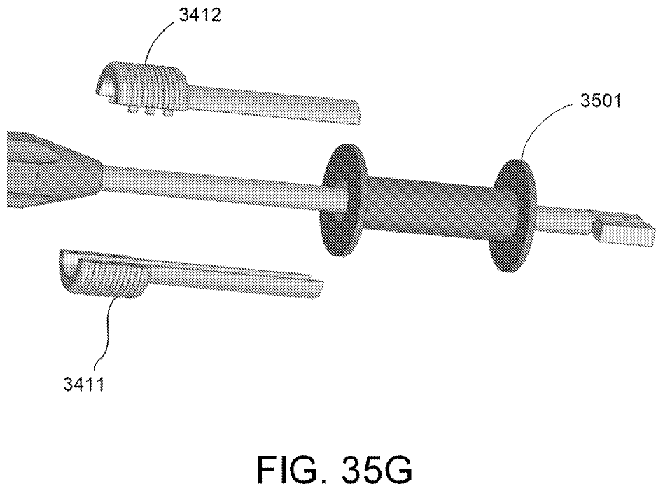

The splitable cannula system 3400 may be used with a conventional implantable access port. Conventional access ports may be compliant, double-flanged devices that may be used to access arthroscopic workspaces. Use of the splitable cannula system 3400 is described below with respect to FIGS. 35A-35G.

FIGS. 35A-35G illustrate different configurations of the splitable cannula system 3400 of FIG. 34 while being used to deliver implant material to an implant region. FIG. 35A shows the splitable cannula system 3400 and an implantable access port 3501. While in this initial configuration, the user may insert the splitable cannula system 3400 into the implantable access port 3501 already inserted into the patient. The patient is not shown for clarity.

FIG. 35B shows the splitable cannula system 3400 inserted into the implantable access port 3501. In some examples, a conical end 3432 of the removable obturator 3430 may assist in the insertion of the splitable cannula system 3400 into the implantable access port 3501. Next, in FIG. 35C the removable obturator (not shown) may be removed from the splitable cannula 3410. Therefore, the splitable cannula 3410 may remain inserted into the implantable access port 3501.

FIG. 35D shows implant material attached to the end of an implant delivery system. The implant material is brought toward the splitable cannula 3410. FIG. 35E shows the implant material guided through the splitable cannula 3410 and the implantable access port 3501. In FIG. 35F, the splitable cannula 3410 is withdrawn from the implantable access port 3501 while the implant material remains in the patient (and through the implantable access port 3501). In some examples, a distal end of the splitable cannula 3410 may be grasped and moved proximally away from the implantable access port. FIG. 35G shows the first cannula section 3411 and the second cannula section 3412 separated into two or more sections while the implant material remains inserted through the implantable access port 3501 and in the patient. In some cases, separation of the cannula sections may overcome the removable fixation (not shown). The separated cannula section 3411 and 3412 may easily be removed from around the implant delivery system.

Any of the apparatuses, including access ports and cannulas described herein, may be adapted to help fold or collapse the graft as it is inserted into the lumen of the access port, cannula, etc. For example, in some cases the apparatus may include a funnel-shape into which the implant delivery system is inserted once the graft is held within the jaws at the distal end of the implant delivery system. The funnel may curve or bend the graft as it is inserted into lumen, to prevent over ending (and cracking or breaking) the graft. In some cases the graft may be curved or bent sufficiently so that it may be driven through the narrower-diameter lumen of the cannula or port.

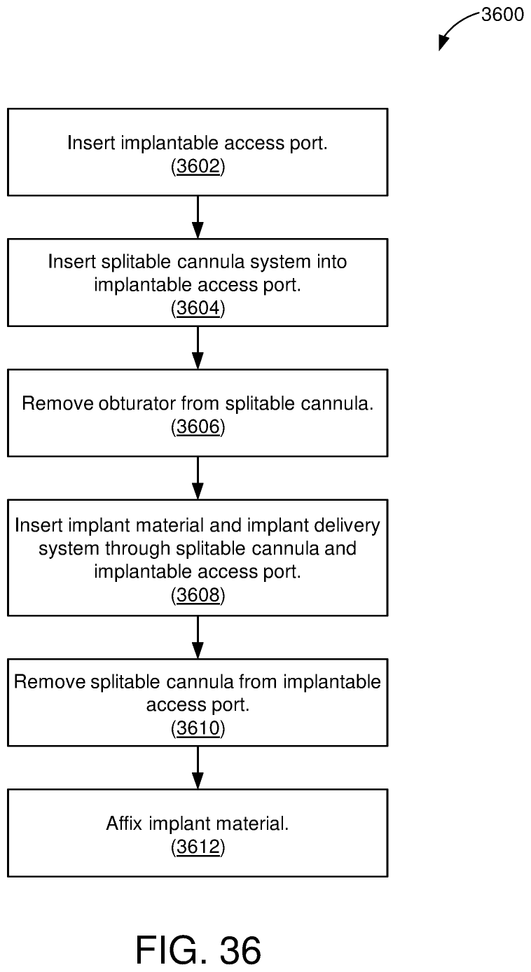

FIG. 36. Is a flowchart showing an example method 3600 for delivering implant material into an implant region, in accordance with some examples. The method 3600 is described below with respect to the splitable cannula system 3300 of FIG. 33, however, the method 3600 may be performed by any other suitable system or device.

The method 3600 begins in block 3602 as an implantable access port is inserted into the patient. The implantable access port can be any feasible port including, but not limited to, the implantable access port 3501 of FIG. 35. Next, in block 3604 the splitable cannula system 3300 is inserted into the implantable access port 3501. For example, the removable obturator 3330 may be used to guide and insert the splitable cannula system 3300 into the implantable access port 3501.

Next, in block 3606 the removable obturator 3330 is removed from the splitable cannula 3310. In block 3608 the implant material and, in some cases, a portion of the implant delivery system are inserted through the splitable cannula 3310 and the implantable access portal 3501. For example, implant material may be attached to the implant delivery system. The user may insert both the implant material and a shaft of the implant delivery system into the splitable cannula 3310. This action may place the implant material in or near an implant region.

Next, in block 3610 the splitable cannula 3310 is removed from the implantable access port 3501. For example, while the implant material and the implant delivery system remain within the implantable access port 3501, the splitable cannula 3310 may be moved proximally allowing the splitable cannula 3310 to be separated and removed. Next in block 3612 the implant material may be affixed or attached to the patient. For example, the implant material may be moved forward through the implantable access port into the implant region. The user may then suture or otherwise attach the implant material to the patient.

It should be appreciated that all combinations of the foregoing concepts and additional concepts discussed in greater detail below (provided such concepts are not mutually inconsistent) are contemplated as being part of the inventive subject matter disclosed herein and may be used to achieve the benefits described herein.

The process parameters and sequence of steps described and/or illustrated herein are given by way of example only and can be varied as desired. For example, while the steps illustrated and/or described herein may be shown or discussed in a particular order, these steps do not necessarily need to be performed in the order illustrated or discussed. The various example methods described and/or illustrated herein may also omit one or more of the steps described or illustrated herein or include additional steps in addition to those disclosed.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive, and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately.” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1. An apparatus for delivering implant material percutaneously to a patient, the apparatus comprising:
a handle having a palm region;
a first finger control coupled to the handle and a second finger control coupled to the handle;
a shaft extending distally from the handle; and
a first distally-extending engagement jaw and a second distally-extending engagement jaw extending from a distal end of the shaft and configured to secure a tissue graft between the first and second distally-extending engagement jaws, wherein actuating the first finger control retracts the first distally-extending engagement jaw proximally and wherein actuating the second finger control retracts the second distally-extending engagement jaw proximally.

2. The apparatus of claim 1, wherein the handle is configured to be held in one hand so that the first finger control and the second finger control may be actuated with the same hand that is holding the handle.

3. The apparatus of claim 1 further comprising a lock preventing retraction of the first distally-extending engagement jaw and the second distally-extending engagement jaw.

4. The apparatus of claim 3, further comprising a lock release configured to release the lock.

5. The apparatus of claim 1, wherein the first distally-extending engagement jaw comprises an end configured to engage with the graft and wherein the first distally-extending engagement jaw is coupled to a rod configured to couple to the first finger control.

6. The apparatus of claim 1, wherein the first distally-extending engagement jaw and the second distally-extending engagement jaw have a flat surface configure to distribute a gripping force exerted by the first distally-extending engagement jaw and the second distally-extending engagement jaw over a surface of the graft.

7. The apparatus of claim 1, wherein the first distally-extending engagement jaw and the second distally-extending engagement jaw each comprises a rod.

8. The apparatus of claim 1, wherein the handle is at least one of a cylindrical handle, a pistol-grip handle, or a t-handle.

9. A method for delivering a graft material into an implant region, the method comprising:
inserting a cannula into a patient, wherein a distal end of the cannula is positioned near the implant region;
securing the graft material between a first distally-extending engagement jaw and a second distally-extending engagement jaw of an applicator, wherein the engagement jaws extend beyond a distal end of a shaft of the applicator;
inserting the graft material in a curved or bent configured in which the graft material is curved or bent around the first distally-extending engagement jaw and the second distally-extending engagement jaw through the cannula;
positioning the graft material near the target site;
retracting the first distally-extending engagement jaw proximally into the shaft and positioning the graft against the target tissue while holding the graft in position against the target tissue with the second distally-extending engagement jaw;
securing the graft to the target tissue and retracting the second distally-extending engagement jaw proximally.

10. The method of claim 9, wherein retracting the first distally-extending engagement jaw comprises actuating a finger control on the handle of the applicator.

11. The method of claim 9, wherein the shaft is coupled to a handle and the shaft extends distally away from the handle.

12. The method of claim 9, wherein the first and/or second distally-extending engagement jaw comprises a planar jaw member.

13. The method of claim 9, wherein the first and/or second distally-extending engagement jaw comprises a rod.

14. The method of claim 9, wherein the second distally-extending engagement jaw provides pressure to the implant material while the implant material is being affixed to the patient.

15. A method of repairing a rotator cuff, the method comprising:
removing a region of a cortical layer of a humerus to form a decorticated surface;
positioning a graft over the decorticated surface;
temporarily securing a mineralized face of the graft on the decorticated surface with a removable securement, wherein the mineralized face has a thickness that is less than a demineralized face of the graft that is adjacent to the mineralized face;
suturing a tendon against the demineralized face of the graft with one or more sutures that do not pass through the graft; and
removing the removeable securement from the graft so that the graft is held against the decorticated surface by just the surgically repaired tendon.

16. The method of claim 15, wherein the removable securement comprises one or more prongs.

17. The method of claim 15, wherein temporarily securing comprises passing one or more prongs of the removable securement through the graft and into the decorticated surface.

18. The method of claim 15, wherein removing the removable securement comprises removing the removable securement from between the graft and the tendon, leaving the tendon sutured against the graft.

19. The method of claim 15, wherein temporarily securing the graft comprises driving one or more prongs of the removeable securement through the decorticated surface to create one or more marrow vents within the humerus into a marrow material.

20. The method of claim 19, wherein removing the removeable securement includes removing the one or more prongs from the decorticated surface, thereby causing the marrow material to exit the one or more marrow vents and into the graft.

21. The method of claim 15, wherein removing the removeable securement comprises removing the one or more prongs from the decorticated surface by pulling a tether attached to the removeable securement.

22. The method of claim 21, wherein pulling the tether comprises pulling the tether in a non-perpendicular direction with respect to a long axis of the one or more prongs.

23. The method of claim 21, wherein pulling the tether causes the removeable securement to pivot such that the one or more prongs exit corresponding one or more opening within the decorticated surface.

24. The method of claim 15, further comprising forming microfractures in the decorticated surface.

25. The method of claim 15, wherein suturing a tendon includes anchoring the one or more tendon-securing sutures to the humerus outside of the decorticated region.

26. The method of claim 15, wherein securing the tendon comprises crossing the one or more tendon-securing sutures over the tendon.

27. The method of claim 15, wherein suturing the tendon comprises suturing the tendons of one or more of: the supraspinatus muscle, infraspinatus muscle teres minor muscle or the subscapularis muscle.

28. A method of repairing a tissue, the method comprising:
removing a region of a cortical layer of a bone to form a decorticated surface;
positioning a graft over the decorticated surface;
temporarily securing a mineralized face of the graft on the decorticated surface with a removable securement, wherein the mineralized face has a thickness that is less than a demineralized face of the graft that is adjacent to the mineralized face;
suturing a tendon against the demineralized face of the graft with one or more sutures that do not pass through the graft; and
removing the removeable securement from the graft so that the graft is held against the decorticated surface by just the surgically repaired tendon.

29.-89. (canceled)
